(19) United States
(12) Patent Application Publication
(10) Pub. No.: US 2023/0149427 A1
(43) Pub. Date: May 18, 2023

(54) COMPOSITIONS COMPRISING COCRYSTALS OF ACETYLSALICYLIC ACID AND THEANINE WITH TROMETHAMINE AND METHODS OF USE

(71) Applicant: Theaprin Pharmaceuticals, Inc., Hauppauge, NY (US)

(72) Inventors: Philip V. FELICE, Setauket, NY (US); Harry G. BRITTAIN, Milford, NJ (US); Paul WABNITZ, Hallett Cove SA (AU)

(21) Appl. No.: 18/077,350

(22) Filed: Dec. 8, 2022

Related U.S. Application Data
(63) Continuation of application No. PCT/US2022/026095, filed on Apr. 25, 2022.
(60) Provisional application No. 63/331,902, filed on Apr. 18, 2022.
(60) Provisional application No. 63/257,607, filed on Oct. 20, 2021.
(60) Provisional application No. 63/257,606, filed on Oct. 20, 2021.
(60) Provisional application No. 63/180,121, filed on Apr. 27, 2021.
(60) Provisional application No. 63/180,120, filed on Apr. 27, 2021.

(51) Int. Cl. A61K 31/616; A61K 31/198; A61K 45/06; A61K 9/08; A61K 47/18; A61K 33/30

(52) U.S. Cl. CPC A61K 31/616; A61K 31/198; A61K 45/06; A61K 9/08; A61K 47/18; A61K 33/30

(57) ABSTRACT

Compositions comprising cocrystals of acetylsalicylic acid and theanine that have been formulated with tromethamine are provided. Methods of preparing the compositions and methods of using the compositions with optional zinc or dipyridamole treatments are also provided.

### REFERENCED TO RELATED APPLICATIONS

This application is a continuation of International Application No. PCT/US2022/026095, which designated the United States and was filed on Apr. 25, 2022, published in English, which claims priority to U.S. Provisional Patent Application No. 63/180,121, filed Apr. 27, 2021, U.S. Provisional Patent Application No. 63/180,120, filed Apr. 27, 2021, U.S. Provisional Patent Application No. 63/257,606, filed Oct. 20, 2021, U.S. Provisional Patent Application No. 63/257,607, filed Oct. 20, 2021, and U.S. Provisional Patent Application No. 63/331,902, filed on Apr. 18, 2022, the contents of each of which are herein incorporated by reference in their entirety.

### FIELD OF THE INVENTION

The disclosure relates to a cocrystal composition of acetylsalicylic acid and theanine cocrystal, further comprising tromethamine as buffer, for the treatment of COVID-19 disease and its related disorders.

### BACKGROUND OF THE INVENTION

In the past two decades, severe acute respiratory syndrome coronavirus (SARS-CoV) and Middle East respiratory syndrome coronavirus (MERS-CoV) were transmitted from animals to humans, causing severe respiratory diseases SARS and MERS in endemic areas [1]. In December 2019, another coronavirus was discovered in patients with infectious respiratory disease in Wuhan, Hubei province, China, to have the ability for human-to-human transmission [1]. The disease, now termed coronavirus disease 2019 (COVID-19), has spread rapidly all over the world, resulting in a pandemic. COVID-19 is induced by the pathogenic Severe Acute Respiratory Syndrome coronavirus 2 (SARS-CoV-2) [1]. SARS-CoV-2 is a single-stranded RNA virus encoding 16 nonstructural proteins (1-16), 8 accessory proteins (ORF3a, 6, 7a, 7b, 8, 9b, 9c, and 10), and 4 structural proteins known as S (spike), E (envelope), M (membrane), and N (nucleocapsid) proteins [2,3]. The spike glycoprotein is responsible for recognition of host cell membrane receptors ACE2 and TMPRSS2 and for mediating fusion with the host cell membrane [2,4].

Coronavirus infects vascular endothelial cells via angiotensin-converting enzyme 2, which is expressed at a high level on pneumocytes and endothelial cells [5,6]. These findings can explain the clinical presentation of severe COVID-19 which is characterized by ARDS, shock, and coagulopathy [5,7,8] where the coagulopathy is a hypercoagulable state.

Zhou et al. [1,9] and Hoffmann et al. [1,4] identify ACE2 as a SARS-CoV-2 receptor, and the latter show its entry mechanism depends on cellular serine protease TMPRSS2. These results may explain pro-inflammatory cytokine release via the associated angiotensin II pathway and a possible therapeutic target via the IL-6-STAT3 axis [1,4,9].

The information about coagulopathy in COVID-19 is still evolving, however, evidence shows that thrombotic coagulation disorder is quite common in severe cases [5]. The incidence of thrombocytopenia is relatively low compared with septic shock, whereas the D-dimer is more sensitive than other coagulation markers and more valuable for the severity measure [5]. Compared with the high incidence of thrombotic events, bleeding complication is considerably rare in COVID-19, and therefore, standard anticoagulant therapy can strongly be recommended [5]. COVID-19 infection causes an intense inflammatory reaction [5]. It is speculated that the dysregulated immune responses orchestrated by inflammatory cytokines, lymphocyte cell death, hypoxia, and endothelial damage are involved [5]. The lung tissue damages are induced by uncontrolled activation of lymphocytes and possibly neutrophil activation (neutrophil extracellular [symbol] traps formation) [5]. Increased pulmonary production of platelets is also involved in the defense process [5]. Inflammatory injury to the alveoli epithelium results in diffuse alveolar damage and in the process, pro-inflammatory mediators are released. In the damaged lung, the virulence of COVID-19 or unabated inflammatory reaction causes pulmonary microthrombi, endothelial damage, and vascular leakage [5] resulting in ARDS (Acute Respiratory Distress Syndrome). The host intends to control the thrombi formation by vigorous fibrinolysis because lung has high fibrinolytic capacity [5]. The fibrin degraded fragment (D-dimer) spills into the blood and is detected in the blood samples [5]. There remains a need for inhibiting the viral replication of coronavirus, treating and/or alleviating the symptoms of COVID-19, and preventing the progression of COVID-19.

Currently, the antiviral VEKLURY (Remdesivir) a nucleoside analogue is the only drug approved by the FDA for the treatment of COVID-19.

Anti-IL-6 drugs are being widely used experimentally and as off-label therapy for patients with COVID-19 who are sick and deteriorating but have a reasonable chance of recovering, but they are still unproven and unapproved for this use [10]. A variety of experimental therapies targeting the hyper-inflammatory phase of COVID-19 are now being applied in hospitals around the world [10]. Among the most widely used treatments are monoclonal antibodies targeting interleukin-6 (IL-6) or the IL-6 receptor [10]. Monoclonal antibodies that are currently being used to treat IL-6 in COVID-19 patients carries with it the potential for immunosuppression and opportunistic infections with long term use [10,11]. The infectious complications of IL-6 inhibition in the acute setting will not be known until a large number of patients is analyzed [10]. Other adverse effects of monoclonal antibodies include gastrointestinal perforations [10,12]. IL-6 inhibitors are associated with a higher incidence of gastrointestinal perforations (1 to 2 per 1,000 patient-years compared with tumor necrosis factor inhibitor use) [10,12]. Other adverse effects include laboratory abnormalities. Neutralization of IL-6 can be associated with leukopenia, thrombocytopenia, and aminotransferase elevations [10]. Chronic use of anti-IL-6 agents is also associated with perturbations of serum lipids, though this is not a concern in the acute setting [10]. In the most severe forms of the disease, the course is often attended by a syndrome that has been described as “cytokine storm,” with some features shared with macrophage activation syndrome [10,13]. Cytokines like IL-6 are inflammatory proteins that act as signaling molecules between cells. During a cytokine storm there is an over production of cytokines in response to infection or non-infectious etiologies. This surge in cytokines results in recruitment of more immune cells creating a hyper-inflammatory response and release of free radicals resulting in permanent damage to surrounding tissue. Cytokine storm is essentially the body's reaction to increase immune activity that is left unchecked. C-reactive protein (CRP), a key acute-phase reactant, can be viewed as a downstream secondary messenger for IL-6 and thus is a reliable biomarker of its activity [10,14]. There remains a need for targeting IL-6 in the treatment of cytokine storm in COVID-19.

### SUMMARY OF THE INVENTION

The present disclosure provides compositions comprising cocrystals of acetylsalicylic acid and L-theanine formulated with tromethamine. Methods of preparing the compositions and methods of using the compositions to treat diseases, such as diseases related to COVID-19, are also provided. Combination therapies with zinc or dipyridamole are also provided.

In some embodiments, the theanine enantiomer may be L-theanine, D-theanine, or D,L-theanine.

In some embodiments, the theanine enantiomer may be an alpha variant of theanine or a beta variant of theanine.

In some embodiments, the alpha variant of theanine may be L-northeanine, D-northeanine, DL-northeanine, L-homotheanine, D-homotheanine, DL-homotheanine, L-bishomotheanine, D-bishomotheanine, or D,L-bishomotheanine.

In some embodiments, the alpha variant of theanine is a homologous analog of theanine.

In some embodiments, the alpha variant of theanine contains a functional group such as linear, cyclic, or branched alkyl groups and derivatives thereof; linear, cyclic, or branched alkenyl groups and derivatives thereof; or aromatic radicals and derivatives thereof. In some embodiments, the aromatic radicals are aryl radicals.

In some embodiments, the theanine enantiomer is a racemic mixture of a beta variant of theanine containing a functional group such as linear, cyclic, or branched alkyl groups and derivatives thereof; linear, cyclic, or branched alkenyl groups and derivatives thereof; or aromatic radicals and derivatives thereof. In some embodiments, the aromatic radicals are aryl radicals.

In some embodiments the theanine enantiomer is an S enantiomer of a beta variant of theanine containing a functional group such as linear, cyclic, or branched alkyl groups and derivatives thereof; linear, cyclic, or branched alkenyl groups and derivatives thereof; or aromatic radicals and derivatives thereof. In some embodiments, the aromatic radicals are aryl radicals.

In some embodiments, the theanine enantiomer is an R enantiomer of a beta variant of theanine containing a functional group such as linear, cyclic, or branched alkyl groups and derivatives thereof; linear, cyclic, or branched alkenyl groups and derivatives thereof; or aromatic radicals and derivatives thereof. In some embodiments, the aromatic radicals are aryl radicals.

In some embodiments, in addition to L-theanine, other analogues include D-theanine, racemic theanine or D, L-theanine and its congeners including beta and reverse beta amino acid forms, shortened or nor-theanine (aspartic acid analogue), and the lengthened homo-theanines and their isomers. Further, gamma alkylamido analogues extend a full range of molecular property for drug cocrystals.

In one embodiment, the disclosure relates to cocrystal compositions of a drug from a specified drug class, and the enantiomers, L- and D-isomers, D, L-racemic mixture, S- and R-isomers, S, R-racemic mixtures, all rotamers, tautomers, salt forms, and hydrates of the alpha and beta amino acids of theanine in which the N-substituted functional R1-group [C4 or gamma-CH2-C(O)—NR1] may contain linear, cyclic, or branched alkyl groups and derivatives thereof; linear, cyclic or branched alkenyl groups and derivatives thereof; and aromatic radicals (which may be aryl radicals) and derivatives thereof making up all the analogue forms of theanine.

In one embodiment, the disclosure relates to a method of treating a disease or disorder in a subject in need thereof, comprising administering to the subject a dosage form of the disclosure.

In some embodiments, a composition comprising water soluble aspirin and L-theanine, together with tromethamine are administered via intravenous infusion.

In some embodiments, the composition comprising the water-soluble aspirin/L-theanine cocrystal together with tromethamine inhibits the formation of pro-inflammatory cytokines in subjects with COVID-19 disease. In some embodiments, the subjects also receive treatment with zinc or dipyridamole.

Zinc, as used herein, refers to a composition comprising zinc. Zinc may be in the form of zinc chloride (ZnCl2), zinc sulfate (ZnSO4), zinc nitrate (Zn(NO3)2), or a hydrate or salt thereof. Zinc may be administered via intravenous infusion.

In some embodiments, zinc inhibits both the proteolytic processing of replicase polyproteins and the RNA-dependent RNA polymerase (RdRp) activity.

In some embodiments, zinc has a synergistic effect with the water-soluble aspirin/L-theanine cocrystal, tromethamine formulation in the treatment of the intense inflammatory response related to cytokine storm in COVID-19 disease.

In some embodiments, zinc has a synergistic immuno-modulatory effect with the water-soluble aspirin/L-theanine cocrystal, tromethamine formulation to downregulate interleukin-6 in the treatment of cytokine storm in COVID-19 disease.

In some embodiments, zinc has a synergistic anti-viral effect with the water-soluble aspirin/L-theanine cocrystal, tromethamine formulation to downregulate interleukin-6 in the treatment of cytokine storm in COVID-19 disease.

In some embodiments, dipyridamole together with the water-soluble aspirin/L-theanine cocrystal, tromethamine formulation is used for the treatment of COVID-19 disease.

In some embodiments, dipyridamole has a synergistic effect with the water-soluble aspirin/L-theanine cocrystal, tromethamine formulation in the treatment of COVID-19 coagulopathy where the coagulopathy is a hypercoagulable state.

In some embodiments, dipyridamole has a synergistic effect with the water-soluble aspirin/L-theanine cocrystal, tromethamine formulation in the treatment of the intense inflammatory response related to cytokine storm in COVID-19 disease.

In some embodiments, dipyridamole has a synergistic immuno-modulatory effect with the water-soluble aspirin/L-theanine cocrystal, tromethamine formulation in the treatment of COVID-19 disease.

In some embodiments, dipyridamole has a synergistic anti-viral effect with the water-soluble aspirin/L-theanine cocrystal, tromethamine formulation in the treatment of COVID-19 disease.

In some embodiments, dipyridamole has a synergistic effect with the water soluble aspirin/L-theanine cocrystal, tromethamine formulation in the treatment of COVID-19 coagulopathy, where the coagulopathy is a hypercoagulable state resulting in an endotheliopathy wherein the endotheliopathy, consist of, but limited to endothelial damage.

In some embodiments, dipyridamole together with the water soluble aspirin/L-theanine cocrystal, tromethamine formulation protects against endothelial damage in COVID-19 disease.

In some embodiments, dipyridamole together with the water-soluble aspirin/L-theanine cocrystal, tromethamine formulation reduces pulmonary hypertension without significantly affecting systemic blood pressure in COVID-19 disease.

In some embodiments, dipyridamole together with the water-soluble aspirin/L-theanine cocrystal, tromethamine formulation is expected to improve lung perfusion by improving the alveolar dead space to tidal volume ratio in ventilator dependent patients with COVID-19 coagulopathy, where the coagulopathy is a hypercoagulable state affecting the alveoli.

In some embodiments, dipyridamole together with the water-soluble aspirin/L-theanine cocrystal, tromethamine formulation is expected to improve the PaO2/FIO2 ratio in ventilator dependent COVID-19 patients with ARDS and coagulopathy, where the coagulopathy is a hypercoagulable state affecting the alveoli.

In some embodiments, dipyridamole together with the water-soluble aspirin/L-theanine cocrystal, tromethamine formulation increases myocardial perfusion in COVID-19 disease.

In some embodiments, dipyridamole together with the water-soluble aspirin/L-theanine cocrystal, tromethamine formulation can reduce cardio-respiratory vasoconstriction due to hypoxia without significantly compromising hemodynamic stability in hypotensive patients in COVID-19 disease.

In some embodiments, dipyridamole together with the water-soluble aspirin/L-theanine cocrystal, tromethamine formulation inhibits the formation of pro-inflammatory cytokines in COVID-19 disease.

In some embodiments, dipyridamole inhibits the formation of pro-inflammatory cytokines and together with ASA protects against endothelial damage.

In some embodiments, dipyridamole has anticoagulant properties.

In some embodiments, dipyridamole inhibits platelet aggregation and causes vasodilation in COVID-19 disease.

In some embodiments, dipyridamole has broad spectrum antiviral activity, particularly efficacious against the positive-stranded RNA viruses in COVID-19 disease.

In some embodiments, dipyridamole suppresses inflammation and promotes mucosal healing in COVID-19 disease.

In some embodiments, dipyridamole may prevent acute injury and progressive fibrosis of the lung, heart, liver, and kidney in COVID-19 disease.

In some embodiments, dipyridamole improves the coagulation profiles in COVID-19 coagulopathy, where the coagulopathy is a hypercoagulable state and where the adverse coagulation profile consists of elevated D-Dimer levels, elevated fibrinogen, elevated prothrombin time, and elevated platelet counts.

In some embodiments, dipyridamole is administered via the oral or sublingual routes.

In some embodiments, dipyridamole consists of oral solids (tablets, oral disintegrating tablets), oral liquids, clear homogeneous solutions, suspensions, or powders.

In some embodiments, the amount of dipyridamole in the oral dosage form together with the water soluble aspirin/L-theanine cocrystal formulation with tromethamine is between 25 mg and 600 mg.

Accordingly, it is an object of the disclosure to provide a composition comprising cocrystals of aspirin and L-theanine, with tromethamine to downregulate IL-6 in the treatment of cytokine storm in subjects with COVID-19 disease. In some embodiments, the subjects also receive treatment with zinc or dipyridamole.

In one embodiment, the disclosure relates to a method of treating an acute inflammatory disease associated with COVID-19 disease in subjects in need thereof with a composition comprising cocrystals of aspirin and L-theanine, with tromethamine. Optionally, the subjects also receive treatment with zinc or dipyridamole.

In one embodiment, the disclosure relates to a method of treating a chronic inflammatory disease associated with COVID-19 disease in subjects in need thereof with a composition comprising cocrystals of aspirin and L-theanine, with tromethamine. Optionally, the subjects also receive treatment with zinc or dipyridamole.

In one embodiment, the disclosure relates to a method of treating respiratory acidosis associated with COVID-19 disease in subjects in need thereof with a composition comprising cocrystals of aspirin and L-theanine, with tromethamine. Optionally, the subjects also receive treatment with zinc or dipyridamole.

In one embodiment, the disclosure relates to a method of treating metabolic acidosis associated with COVID-19 disease in subjects in need thereof with a composition comprising cocrystals of aspirin and L-theanine, with tromethamine. Optionally, the subjects also receive treatment with zinc or dipyridamole.

In one embodiment, the disclosure relates to a method of treating lactic acidosis associated with COVID-19 disease in subjects in need thereof with a composition comprising cocrystals of aspirin and L-theanine, with tromethamine. Optionally, the subjects also receive treatment with zinc or dipyridamole.

In one embodiment, the disclosure relates to a method of treating mixed respiratory acidosis and metabolic acidosis or mixed respiratory acidosis and lactic acidosis as a result of COVID-19 disease in subjects in need thereof with a composition comprising cocrystals of aspirin and L-theanine, with tromethamine. Optionally, the subjects also receive treatment with zinc or dipyridamole.

In one embodiment, the disclosure relates to a method of downregulating interleukin-6 in the treatment of cytokine storm in COVID-19 disease in subjects in need thereof with a composition comprising cocrystals of aspirin and L-theanine, with tromethamine. Optionally, the subjects also receive treatment with zinc or dipyridamole.

In one embodiment, the disclosure relates to a method of treating the intense inflammatory reaction associated with COVID-19 disease in subjects in need thereof with a composition comprising cocrystals of aspirin and L-theanine, with tromethamine. Optionally, the subjects also receive treatment with zinc or dipyridamole.

In one embodiment, the disclosure relates to a method of treating the intense inflammatory reaction, where the inflammatory reaction is a cytokine storm consisting of dysregulated immune responses orchestrated by inflammatory cytokines, lymphocyte cell death, hypoxia, and endothelial damage associated with COVID-19 disease in subjects in need thereof with a composition comprising cocrystals of aspirin and L-theanine, with tromethamine. Optionally, the subjects also receive treatment with zinc or dipyridamole.

In some embodiments, compositions comprising water soluble cocrystals of aspirin and L-theanine, together with tromethamine administered via intravenous infusion inhibits IL-6 synthesis through immuno-modulatory cyclo-oxygenase 2 (COX-2) inhibition of prostaglandin E2 and nuclear factor-kappa B antagonism for treating the intense inflammatory reaction, where the inflammatory reaction is a cytokine storm consisting of dysregulated immune responses orchestrated by inflammatory cytokines, lymphocyte cell death, hypoxia, and endothelial damage associated with COVID-19 disease in subjects in need thereof. Optionally, the subjects also receive treatment with zinc or dipyridamole.

In some embodiments, compositions comprising water soluble cocrystals of aspirin and L-theanine, together with tromethamine administered via intravenous infusion inhibits viral replication due to its inhibitory effect on PGE2 and its effect on Type I Interferon Alpha (IFN-α) by inducing the upregulation of numerous genes that activate both innate and adaptive immunity for rapidly controlling viral replication in subjects in need thereof. Optionally, the subjects also receive treatment with zinc or dipyridamole.

In some embodiments, compositions comprising water soluble cocrystals of aspirin and L-theanine, together with tromethamine administered via intravenous infusion inhibits the activity of IκB kinase-β, thereby preventing activation of nuclear factor-KB, which is involved in the pathogenesis of inflammation, in subjects in need thereof. Optionally, the subjects also receive treatment with zinc or dipyridamole.

In some embodiments, compositions comprising water soluble cocrystals of aspirin and L-theanine, together with tromethamine administered via intravenous infusion triggers anti-inflammatory 15-epi-lipoxin A4 and induction of apoptosis of inflammatory cells via the mitogen-activated protein kinase pathway in subjects in need thereof. Optionally, the subjects also receive treatment with zinc or dipyridamole.

In some embodiments, compositions comprising water soluble cocrystals of aspirin with L-theanine, together with tromethamine administered via intravenous infusion inhibits leukocyte accumulation of inflammatory cells by an adenosine-dependent mechanism that is independent of inhibition of prostaglandin synthesis and p105 of NF kappa B in subjects in need thereof. Optionally, the subjects also receive treatment with zinc or dipyridamole.

In some embodiments, compositions comprising water soluble cocrystals of aspirin and L-theanine, together with tromethamine administered via intravenous infusion inhibits IL-6 expression of metallothioneins and α2-macroglobulin by modulation of the pro-inflammatory response by targeting nuclear factor kappa B(NF-κB), a transcription factor that is the master regulator of pro-inflammatory responses for the treatment of COVID-19 cytokine storm, in subjects in need thereof. Optionally, the subjects also receive treatment with zinc or dipyridamole.

In some embodiments, compositions comprising water soluble cocrystals of aspirin and L-theanine, together with tromethamine administered via intravenous infusion inhibits IL-6 mediated activation of STAT3 for the treatment of COVID-19 cytokine storm in subjects in need thereof. Optionally, the subjects also receive treatment with zinc or dipyridamole.

In some embodiments, compositions comprising water soluble cocrystals of aspirin and L-theanine, together with tromethamine administered via intravenous infusion has antioxidant properties neutralizing free radicals for the treatment of COVID-19 cytokine storm in subjects in need thereof. Optionally, the subjects also receive treatment with zinc or dipyridamole.

In one embodiment, compositions comprising water soluble cocrystals of aspirin and L-theanine, together with tromethamine in combination with zinc or dipyridamole have a synergistic effect in the treatment of the intense inflammatory response related to cytokine storm in COVID-19 disease, in subjects in need thereof.

In some embodiments, compositions comprising water soluble cocrystals of aspirin with L-theanine together with tromethamine administered via intravenous infusion are expected to improve the PaO2/FIO2 ratio in ventilator dependent COVID-19 patients with cytokine storm and Acute Respiratory Distress Syndrome (ARDS). Optionally, the patients also receive treatment with zinc or dipyridamole.

In some embodiments, compositions comprising water soluble cocrystals of aspirin with L-theanine together with tromethamine administered via the intravenous infusion are expected to improve the blood oxygen saturation in non-ventilator dependent COVID-19 patients with cytokine storm. Optionally, the patients also receive treatment with zinc or dipyridamole.

In some embodiments, zinc has a synergistic effect with compositions comprising water-soluble aspirin/L-theanine cocrystals together with tromethamine in the treatment of COVID-19 coagulopathy, where the coagulopathy is a hypercoagulable state resulting in an endotheliopathy.

In some embodiments, zinc has a synergistic effect with compositions comprising water soluble aspirin/L-theanine cocrystals together with tromethamine that reduces endothelial damage in COVID-19 disease.

In some embodiments, the amount of aspirin cocrystal in the intravenous dosage form is between 200 mg and 500 mg.

In some embodiments, the amount of L-theanine cocrystal in the intravenous dosage form is between 200 mg and 500 mg.

In some embodiments, the amount of aspirin cocrystal in the intravenous dosage form is between 100 mg and 1.5 g.

In some embodiments, the amount of L-theanine cocrystal in the intravenous dosage form is between 100 mg and 1.5 g.

In some embodiments, the amount of tromethamine in the intravenous dosage form is between 50 mg to 500 mg, such as between 50 mg to 100 mg, 100 mg to 200 mg, between 200 mg to 300 mg, between 300 to 400 mg, or between 400 mg to 500 mg.

In some embodiments, the amount of zinc in the intravenous dosage form is between 0.1 mg and 100 mg, such as between 0.1 mg to 0.5 mg, 0.5 mg to 1 mg, 1 mg to 2 mg, 2 mg to 3 mg, 3 mg to 4 mg, 4 mg to 5 mg, 5 mg to 10 mg, 10 mg to 20 mg, between 20 mg to 30 mg, between 30 mg to 40 mg, between 40 mg to 50 mg, between 50 mg to 60 mg, between 60 mg to 70 mg, between 70 mg to 80 mg, between 80 mg to 90 mg, between 90 mg to 100 mg.

In some embodiments, the wt. % of aspirin in the intravenous cocrystal formulation is between about 10% to about 50%.

In some embodiments, the wt. % of L-theanine in the intravenous cocrystal formulation is between about 10% to about 50%.

In some embodiments, the wt. % of tromethamine in the intravenous formulation is between about 10% to about 50%.

In some embodiments, the wt. % of zinc in the intravenous formulation is between about 1% to about 10%.

In some embodiments, the molar ratio of aspirin and L-theanine in the cocrystal is about 1:1. In some embodiments, the molar ratio of aspirin, L-theanine, and tromethamine is 1:1:z:y, wherein z is between about 1 and about 5 (such as between about 1.0 and about 1.9, between about 2.0 and about 2.9, between about 3.0 and about 3.9, or between about 4.0 and about 4.9).

In some embodiments, the pH of the formulation is between about 5.5 and about 8.0, such as between 5.5 to 6.0, between 6.0 to 6.5, between 6.5 to 7.0, between 7.0 to 7.5, and between 7.5 to 8.0. In some embodiments, the pH of the formulation is between about 6.8 to about 7.4. In some embodiments, the pH of the formulation is between about 5.9 to about 7.8. In some embodiments, the pH of the formulation is about 7.0.

In some embodiments, the osmolality of the intravenous formulation is greater than 260 mOsm/kg and less than 601 mOsm/kg.

### DETAILED DESCRIPTION OF THE INVENTION

### Interleukin-6 (IL-6)

IL-6 is recognized by two membrane receptors, IL-6Rα receptor and glycoprotein 130 (gp130) [15]. Binding of IL-6 to its receptors forms an active membrane protein complex and leads through gp130 to the activation of the Janus Kinase/Signal Transducer and Activation of Transcription (JAK/STAT) cascade and the Mitogen-Activated Protein Kinase (MAPK) cascade. IL-6 signaling is terminated by tyrosine phosphatases, Suppressor of Cytokine Signaling (SOCS) proteins and Protein Inhibitor of Activated STAT (PIAS) proteins [15]. The balance between signaling pathways and suppressors of signaling mediates the final action of IL-6 in cells [15].

IL-6 is important for the development of diseases such as asthma [16], idiopathic pulmonary fibrosis (IPF) [17] and acute respiratory distress syndrome (ARDS) [18].

Human IL-6 is a protein with a molecular weight of 21 kDa-28 kDa [19]. Crystallography X showed that IL-6 is formed by 4 a-helices, arranged as two couples of anti-parallel helices [20]. A structure of human IL-6 can be found in UniProt database entry number P05231.

### Histopathology

Early histopathology reports describe findings of diffuse alveolar damage with profound inflammation, thrombosis, and thrombotic microangiopathy of small vessels and capillaries of the lung [21]. Endothelial cell injury and diffuse microvascular thrombosis suggestive of thrombotic microangiopathy is also reported in extrapulmonary organs and may explain acute onset of multiorgan failure without an otherwise obvious etiology [21,22].

Megakaryocytes within pulmonary capillaries with nuclear hyperchromasia and atypia, as well as neutrophils partially degenerated and entrapped in fibers, suggesting neutrophil extracellular traps, have also been noted [21,23].

Inflammation is known to promote thrombosis through various mechanisms, including activation of the endothelium, platelets, monocytes, and the tissue factor/factor VIIa pathway, as well as altering fibrinolysis and natural anticoagulant pathways (thrombomodulin, proteins C and S, tissue-factor-pathway inhibitor) [21,24,25].

One mechanism of microvascular thrombosis that may be specific for SARS-CoV-2 is its affinity for angiotensin-converting enzyme 2 (ACE2), which is expressed on alveolar epithelial type II cells and various extrapulmonary tissues including endothelial cells. Endothelial cell activation may represent a unique mechanism of COVID-19-mediated microvascular injury, thrombosis, and subsequent multisystem organ failure [21,26,27].

Both pathogens (viruses) and damage-associated molecular patterns (DAMPs) from injured host tissue can activate monocytes. Activated monocytes release inflammatory cytokines and chemokines that stimulate neutrophils, lymphocytes, platelets, and vascular endothelial cells. Monocytes and other cells express tissue factor and phosphatidylserine on their surfaces and initiate coagulation. Healthy endothelial cells maintain their anti-thrombogenicity by expressing glycocalyx and its binding protein antithrombin. Damaged endothelial cells change their properties to procoagulant following disruption of the glycocalyx and loss of anticoagulant proteins [5].

The inflammatory response in monocytes and macrophages has been linked to the production of thrombin, and the inhibition of thrombin activity can be a potential therapeutic approach [5,28]. Previously reported therapeutic candidates studied in sepsis that can modulate the coagulation cascade include antithrombin and recombinant thrombomodulin. Both agents are also expected to suppress the excess inflammation and thereby inhibit the formation of “immunothrombus” [5,29].

Paradoxical rise in platelet count cannot be clearly explained, but the involvement of proinflammatory cytokines is suspected in coronavirus infection [5,30]. The “cytokine storm” of dysregulated proinflammatory cytokines such as interleukin (IL)-1β and IL-6 stimulates the proliferation of the megakaryocytes, which causes the thrombocytosis [5].

### “Hyper-Innate-Immunity” and Severe Covid-19 Disease

Pathogenic viruses, such as SARS-CoV-2 damage the vascular endothelium. This then can lead to an aggressive and prolonged innate response which involves neutrophil recruitment, platelet aggregation, mucous cell hyperplasia, cell leakage, inflammatory exudate and airspace enlargement in the lungs, with a decreased alveolar oxygen transfer leading to respiratory failure. Endothelial damage, and the resultant hypoxic vasoconstriction, further promote cellular destruction and activation of additional pro-inflammatory agents, such as cytokines and can lead to cytokine storm—a common complication of severe COVID-19. In vulnerable populations, platelet aggregation, vasoconstriction and pro-inflammation can also increase the risk of venous stasis and produce a “hyper-prothrombotic” state.

This process in COVID-19 is largely driven by a “hyper-innate immune” response and has been established in previous severe respiratory coronaviruses (MERS-CoV, SARS-CoV) [31]. Cytokine storm, with elevation of interleukin (IL)-2R, IL-6, IL-1β, IL-8, IL-17, granulocyte colony-stimulating factor (G-CSF), tumour necrosis factor-α (TNF-α), IP10, MCP1, and MIP1α, is often seen in severe cases of COVID-19 and results in lymphopenia due to immune exhaustion [32].

Cytokine storm, endothelial dysfunction, von Willebrand factor elevation, Toll-like receptor activation, and tissue-factor pathway activation are thought to contribute to a hypercoagulable state, reactive oxidant species, and alveolar neutrophil infiltration. Endothelial dysfunction, von Willebrand factor (vWF) elevation, Toll-like receptor activation, and tissue-factor pathway activation [33,34] may induce proinflammatory and procoagulant effects through complement activation and cytokine release [33,35], resulting in a dysregulation of the coagulation cascade with the subsequent formation of intra-alveolar or systemic fibrin clots.

This unique “Coronavirus” cycle of pulmonary damage can be demonstrated in a number of recently published COVID-19 cases studies. Autopsy findings of 12 consecutive COVID-19 deaths in revealed deep vein thrombosis in 7 patients (58%) whereby thromboembolism was not suspected before death [36]. Pulmonary embolism was the direct cause of death in a further 4 patients [36].

Histologic analysis of pulmonary vessels in 7 patients who died from COVID-19 showed widespread thrombosis with microangiopathy and a much higher prevalence of alveolar capillary microthrombi when compared with those who died from influenza-associated respiratory failure [37]. In another multicentre prospective cohort of 150 severe COVID-19 patients with ARDS admitted to ICU, 25 (16.7%) developed pulmonary embolisms and 3 (2%) developed deep vein thrombosis despite prophylactic or therapeutic anticoagulation [38].

A post-mortem analysis of 38 patients who died from COVID-19 demonstrated that the predominant pattern of lung lesions is diffuse alveolar damage [39]. The studies also show the presence of platelet-fibrin thrombi in the small arterial vessels. This may explain the unusual incidence of acute myocardial infarction and large vessel stroke as a presenting feature in young patients [40]. In a retrospective study of 214 hospitalized patients, 5.7% of the severe patients suffered from acute cerebrovascular disease [41]. All of these relevant findings strongly suggest a “hyper-coagulant” state largely driven by a “hyper-innate” immune reaction and a platelet “hyper-activation and aggregation” in COVID-19 patients, especially if critically ill [42].

Aspirin is an ideal therapy to dampen “Hyper-Immunity” of severe COVID-19 disease. Acetylsalicylic acid (ASA or Aspirin) irreversibly inactivates platelet cyclooxygenase, which is responsible for prostaglandin and thromboxane synthesis and irreversibly blocks production of thromboxane. Thromboxane A2 is a potent platelet activator, a promoter of platelet aggregation and importantly of neutrophil recruitment.

Prothrombotic Autoantibodies in Serum from Patients Hospitalized with COVID-19

Antiphospholipid syndrome is an acquired and potentially life-threatening thrombophilia in which patients develop pathogenic autoantibodies targeting phospholipids and phospholipid-binding proteins (aPL antibodies) [43]. These autoantibodies engage cell surfaces, where they activate endothelial cells, platelets, and neutrophils [43,44,45], thereby tipping the blood-endothelium interface toward thrombosis. A key feature of antiphospholipid syndrome is its ability to promote thrombosis in vascular beds of all sizes, including both arterial and venous circuits [43].

Zuo et. al measured eight types of aPL antibodies in serum samples from 172 patients hospitalized with COVID-19. These aPL antibodies included anticardiolipin IgG, IgM, and IgA; anti-β2 glycoprotein I IgG, IgM, and IgA; and a phosphatidylserine/prothrombin (aPS/PT) IgG and IgM. The researchers detected aPS/PT IgG in 24% of serum samples, anticardiolipin IgM in 23% of samples, and aPS/PT IgM in 18% of samples [43]. Antiphospholipid autoantibodies were present in 52% of serum samples using the manufacturer's threshold and in 30% using a more stringent cutoff (>40 ELISA-specific units). The researchers findings suggest that half of patients hospitalized with COVID-19 become at least transiently positive for aPL antibodies and that these autoantibodies are potentially pathogenic [43].

### Vaccine-Induced Thrombotic Thrombocytopenia

Vaccine-induced immune thrombotic thrombocytopenia (VITT) is a severe adverse effect of ChAdOx1 nCoV-19 COVID-19 vaccine (Vaxzevria) and Janssen Ad26.COV2.S COVID-19 vaccine, and it is associated with unusual thrombosis [2]. VITT is caused by anti-platelet factor 4 (PF4) antibodies activating platelets through their FcγRIIa receptors [2]. Antibodies that activate platelets through FcγRIIa receptors have also been identified in patients with COVID-19 [2]. Greinacher, A. et. al, concluded that antibodies against PF4 induced by vaccination do not cross-react with the SARS-CoV-2 spike protein, indicating that the intended vaccine-induced immune response against SARS-CoV-2 spike protein is not the trigger of VITT and that PF4-reactive antibodies found in patients with COVID-19 in this study were not associated with thrombotic complications [2].

### Compounds and Compositions

The present disclosure provides a formulation that downregulates IL-6, reduces the surge of cytokines, alleviates the symptoms, and prevents progression of the COVID-19 disease. Early use of an intravenous composition comprising cocrystals of acetylsalicylic acid (aspirin) and L-theanine, together with tromethamine to downregulate interleukin-6 (IL-6) in the treatment of cytokine storm in COVID-19 disease inhibits viral replication at an early stage in patients with COVID-19, arrests the progression of the disease and prevents the sequelae associated with the most severe forms of the disease. The patients may also receive zinc or dipyridamole treatments.

Therapeutic compounds, such as aspirin, are most stable in a crystalline form, but can display poor aqueous solubilities and slow dissolution rates. These properties impart the tendency to reduce the bioavailability of the active pharmaceutical ingredient (API), thereby slowing absorption.

A cocrystal is a multiple-component crystal, in which two or more molecules associate (but do not bond) on the molecular level in solid crystalline form under ambient conditions [46]. They are attractive to the pharmaceutical industry because they offer opportunities to modify the chemical and/or physical properties of an API without the need to make or break covalent bonds [46]. In pharmaceutical cocrystals, the molecular structure of the API is not changed. This has important implications for streamlined regulatory approval of new forms [46]. By their very nature, APIs, molecules that contain exterior hydrogen-bonding moieties, are predisposed to formation of cocrystals [46].

Pharmaceutical cocrystals represent an alternative to the use of polymorphs, solvatomorphs, and salts as a means to modify dissolution, crystallinity, and hygroscopicity of drug substances. The act of crystallizing two compounds in a single crystal lattice provides a means to improve the physical properties of a given drug substance, since the different lattice energies inherent to the crystal structures of cocrystals most often results in beneficial alterations in physical properties that have a positive effect on the delivery of a drug substance.

The present disclosure provides an aspirin and theanine cocrystal composition, wherein the composition further comprises tromethamine and zinc. In some embodiments, the cocrystal composition comprising aspirin and theanine, together with tromethamine and zinc, is water-soluble. In some embodiments, the cocrystal composition comprising aspirin and theanine, together with tromethamine and zinc are suitable for administration via intravenous infusion.

A water-soluble composition forms an aqueous solution, which is a solution in which water is the dissolving medium or solvent, and which is essentially free of colloidal solids. Dissolved crystals form true solutions and are capable of passing through a semi-permeable membrane as in dialysis, whereas colloids are unable to pass through a semi-permeable membrane. The compositions of the present disclosure form a true solution when dissolved in water, are able to pass through a semi-permeable membrane, and can be used in dialysis. Non-limiting examples of aqueous solutions that may be used in embodiments of the present disclosure include pure water, and the following: D5W, D10W, D50, D5 0.3% NS, D5 0.45% NS, 0.45% NS, D5 0.9% NS, 0.9% NS, D5RL, LR, and NaHCO3.

Solutions formed by dissolving the cocrystal compositions of the present disclosure in water do not contain colloidal particles, and hence, do not exhibit the strong Tyndall effect characteristic of colloidal dispersions.

It should be understood that the term “suitable,” as it is used herein, generally refers to the fact that the solution can be administered intravenously to humans.

### The Theanine Component of the Cocrystal

Theanine (5-N-ethyl-glutamine) is found in green tea leaves Camellia sinensis. Theanine is synthesized in the root of the tea plant and concentrates in the leaves, where sunlight converts theanine into polyphenols [47].

Theanine and its analogues form zwitterions at neutral pH. The ion charges are available to pair through the cationic or protonated alpha amino group with the ortho carboxylate anion of acetylsalicylic acid. This can be further stabilized by hydrogen bonding to the ethylcarboxamide's nitrogen (this ethylamido group is the functional signature of theanine type molecules). Crystals of this ion pair may encapsulate more water molecules than either molecule alone. This gives the complex its easy solvation on dissolution in water or buffer [48].

In some embodiments of the present disclosure, the theanine enantiomer in the L-form acts as a solubility enhancer in the formulation.

In some embodiments, the theanine enantiomer is the L-form. In some embodiments, the theanine enantiomer is the D-form. In some embodiments, the theanine enantiomer is the DL-form.

In some embodiments of the present disclosure, the theanine enantiomer further comprises a carbohydrate functional group thereon. In these embodiments, the carbohydrate functional group may be of the L-configuration or the D-configuration. In these embodiments, the carbohydrates employed may be monosaccharides, disaccharides, trisaccharides, oligosaccharides or polysaccharides.

In some embodiments of the present disclosure, the theanine enantiomer further comprises an amino acid functional group thereon. In certain of these embodiments, the amino acid functional group is a dipeptide.

Non-limiting examples of enantiomers utilized in embodiments according to the present disclosure may include a D-enantiomer of Theanine, D-Glu(NHEt)-OH, 2R enantiomer; an L-enantiomer of Theanine, L-Glu(NHEt)-OH, 2S enantiomer; a DL enantiomer of Theanine, DL-Glu(NHEt)-OH 2R, 2S enantiomers; a D-enantiomer of Theanine, D-Gln(Et)-OH, 2R enantiomer; an L-enantiomer of theanine, L-Gln(Et)-OH, 2R 2S enantiomer; and a DL-enantiomer of theanine, DL-Gln(Et)-OH, 2R, 2S enantiomers. The purity percentages of the D-enantiomers of theanine, D-Glu(NHEt)-OH, 2R enantiomer and D-Gln(Et)-OH, 2R enantiomer; the L enantiomers of theanine, L-Glu(NHEt)-OH, 2S enantiomer and L-Gln(Et)-OH, 2S enantiomer; and the DL-enantiomers of theanine, DL-Glu(NHEt)-OH, 2R, 2S enantiomers and DL-Gln(Et)-OH, 2R, 2S enantiomers in compositions according to embodiments of the present disclosure is 99+%; 99+% 2R enantiomer. The D-enantiomer at 99+%; 99+% ee (2R) is where the first measure is the overall chemical purity (hplc) and where the second measure is ee % (2R) known as the “percent enantiomeric excess.” The % ee is the measure of chiral purity equal to [% R−% S/% R]*100 defined by the ratios of their diasteriomeric derivatives. Purity percentages may range from 90% to 99.99% in any D or L configuration of any theanine or any enantiomer thereof [48].

Embodiments of the present disclosure may include cocrystal compositions of acetylsalicylic acid and alpha variants of L-theanine, acetylsalicylic acid and alpha variants of D-theanine, and acetylsalicylic acid and alpha variants of DL-theanine.

Non-limiting examples of alpha variants of theanine that can be used in embodiments of the present disclosure may include L-northeanine, D-northeanine, DL-northeanine, L-homotheanine, D-homotheanine, DL-homotheanine L-bishomotheanine, D-bishomotheanine, and DL-bishomotheanine, i.e., the respective C−1, C+1, and C+2 homologous analogues of theanine.

According to embodiments of the present disclosure, the L-, D-, DL-alpha amino acids of theanine and their side-chain carbon homologues (nor, homo, and bishomologues) may have a functional R-group, where R1 may contain linear, cyclic, or branched alkyl groups and derivatives thereof; linear, cyclic, or branched alkenyl groups and derivatives thereof; and aromatic radicals and derivatives thereof. In embodiments of the present disclosure, the aromatic radicals may be aryl radicals.

According to the embodiments of the present disclosure the single enantiomers (S and R) and racemic forms (S, R-mixture) of the beta amino acids of theanine may have a functional R-group, where R1 may contain linear, cyclic, or branched alkyl groups and derivatives thereof; linear, cyclic, or branched alkenyl groups and derivatives thereof; and aromatic radicals and derivatives thereof. In embodiments of the present disclosure, the aromatic radicals may be aryl radicals.

Embodiments of the present disclosure may include cocrystal compositions of acetylsalicylic acid and the enantiomers, L- and D-isomers, D, L-racemic mixture, S- and R-isomers, S, R-racemic mixtures, all rotamers, tautomers, salt forms, and hydrates of the alpha and beta amino acids of theanine in which the N-substituted functional R1-group [C4 or gamma-CH2-C(O)—NR1] may contain linear, cyclic, or branched alkyl groups and derivatives thereof; linear, cyclic, or branched alkenyl groups and derivatives thereof; and aromatic radicals and derivatives thereof making up all the analogue forms of theanine. In embodiments of the present disclosure, the aromatic radicals may be aryl radicals.

According to the embodiments of the present disclosure L-theanine has anti-inflammatory, anticoagulant, antiviral, and immunomodulatory effects that are synergistic with the properties of acetylsalicylic acid in the formulation.

L-theanine (N-ethyl-L-glutamine) an amino acid analog of glutamine, is a non-protein amino acid. Being 5-N-ethyl glutamine, theanine differs from glutamine by the CH2-CH3 (ethyl) group replacing hydrogen. The N-ethyl group confers on theanine its active properties. Theanine is hydrolyzed in the kidney to glutamic acid and ethylamine by the enzyme glutaminase [49].

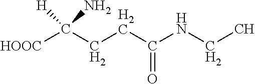

### Chemical Structure of L-Theanine [47]

L-Theanine is an odorless, white crystalline powder that is soluble in water and transparent in solution. L-theanine has a Chemical Abstracts Service (CAS) Registry Number of 3081-61-6 and a GRAS classification (GRAS Notice Number: GRN 000209). L-theanine has the molecular formula C7H14N2O3, molecular weight of 174.20 g/mol, pKa of 2.35, a melting point of 217-218° C. and has an LD50 of greater than 5000 mg/kg in rats [47].

Immunologically, L-theanine's mechanisms of action are multifold. In various animal studies, L-Theanine's anti-inflammatory properties were shown to inhibit the expression of several inflammatory factors including IL-1 β, TNF-α, IL-6, inhibit the expression of pro-inflammatory mediators involved in the nuclear factor-kappa B pathway, such as inducible nitric oxide synthase (iNOS) and matrix metalloproteinase-3, suppress the acute phase response of C-reactive protein levels [50,51], promote the expression of the anti-inflammatory cytokine IL-10 [51], and to inhibit pro-inflammatory PKC/ERK/ICAM-1/IL-33 signaling [52].

Thrombin is a serine protease in the blood plasma that causes coagulation of blood by converting fibrinogen to fibrin. Theanine is a potent inhibitor of thrombin-stimulated thromboxane formation in whole blood [53], and is responsible for theanine's anticoagulant property. Ali et al showed that theanine inhibited thromboxane formation in rabbit whole blood stimulated by thrombin [53]. Inhibition of thromboxane formation by theanine would be expected to significantly reduce the median platelet aggregation inhibition time.

In some embodiments, the theanine is L-theanine. In some embodiments, the amount of L-theanine cocrystal in the intravenous dosage form is between 200 mg and 500 mg. In some embodiments, the wt. % of L-theanine in the intravenous cocrystal formulation is between about 10% to about 50%.

### The Aspirin Component of the Cocrystal

Aspirin (acetylsalicylic acid) inhibits prostaglandin (PG) synthesis by transfer of its acetyl group to a serine residue in the cyclooxygenase (COX) active site. Acetylation of Ser530 inhibits catalysis by preventing access of arachidonic acid substrate in the COX-1 isoenzyme [54], thereby blocking thromboxane A2 synthesis in platelets and reducing platelet aggregation. Thromboxane A2 is a potent platelet activator, a promotor of platelet aggregation, and neutrophil recruitment. Blocking thromboxane A2 synthesis causes platelet aggregation inhibition resulting in a reduced tendency of platelets to clump and reduced neutrophil recruitment.

Aspirin has complex immuno-modulatory effects, mediated by both cyclo-oxygenase (COX) inhibition, and nuclear factor kappa B antagonism. Specifically, IL-6 synthesis is stimulated by prostaglandin E2 via COX-2 [55] and consequently inhibited by aspirin or salicylate metabolites [56]. Upon sensing viral pathogen-associated molecular patterns (PAMPs), macrophages induce a cascade of cytokine responses including type I interferon a (IFN-a) and IFN-β production. Type I IFNs signal through the IFN-a receptor (IFNAR) and induce the upregulation of numerous genes that activate both innate and adaptive immunity for rapidly controlling viral replication [57,58]. Koerner et al demonstrated in the mouse model that expression of type I IFN genes is stringently and that the IFN-ß gene occupies a position at the top of the hierarchy of type I IFN. When IFN-ß is missing, IFN-α synthesis is delayed, thus enhancing the probability that the invading virus can overrun the innate immune response of the host [58]. These studies demonstrate that aspirin can inhibit viral replication due to its inhibitory effect on PGE2 and its effect on Type I Interferon Alpha (IFN-α). Moreover, ASA inhibits the activity of IκB kinase-β, thereby preventing activation of nuclear factor-κB, which is involved in the pathogenesis of inflammation [59]; ASA triggers anti-inflammatory 15-epi-lipoxin A [60] and induction of apoptosis of inflammatory cells via the mitogen-activated protein kinase pathway [61]; and ASA inhibits leukocyte accumulation of inflammatory cells by an adenosine-dependent mechanism that is independent of inhibition of prostaglandin synthesis and p105 of Nuclear Factor kappa B [62]. In a retrospective, observational cohort study of adult patients admitted with COVID-19 to multiple hospitals in the United States between March 2020 and July 2020, Chow J. H, et al demonstrated that aspirin use is associated with decrease mechanical ventilation, ICU admission, and in-hospital mortality in hospitalized patients with COVID-19 [63]. The aspirin utilized in Chow's study was not an aspirin/theanine cocrystal.

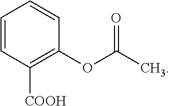 [SMILES: CC(=O)Oc1ccccc1C(=O)O]

### Chemical Structure of Aspirin Cas No. 50-78-2

In some embodiments, the amount of aspirin cocrystal in the intravenous dosage form is between 200 mg and 500 mg. In some embodiments, the wt. % of aspirin in the intravenous cocrystal formulation is between about 10% to about 50%.

### pH Dependent Viral Entry of SARS-CoV-2

The SARS-CoV-2 spike employs mobile receptor-binding domains (RBDs) to engage the human ACE2 receptor and to facilitate virus entry, which can occur through low-pH-endosomal pathways [64].

During endocytosis, at physiological pH, the SARS-CoV-2 spike protein binds ACE2 and that weak folding constraints enable antibodies like CR3022 to bind to the spike protein of the SARS-CoV-2 virion with avidity [64]. A pH-dependent (conformational) switch mediates endosomal positioning of SARS-CoV-2 spike receptor-binding domains [64]. In late endosome-early lysosome where the pH is acidic (pH 5.5-4.5), the spike adopts an all-RBD-down conformation, which provides a potential means of immune evasion from RBD-up-recognizing antibody [64]. Shed antibodies no longer bind the spike protein, which can now be activated by the low-pH protease TMPRSS2 [64]. TMPRSS2 then cleaves the spike protein which allows the SARS-CoV-2 virion to enter the cytosol.

The SARS-CoV-2 spike employs mobile receptor-binding domains (RBDs) to engage the human ACE2 receptor and to facilitate virus entry, which can occur through low-pH-endosomal pathways [64]. During endocytosis, at physiological pH, the SARS-CoV-2 spike protein binds ACE2 and that weak folding constraints enable antibodies like CR3022 to bind to the spike protein of the SARS-CoV-2 virion with avidity [64]. A pH-dependent (conformational) switch mediates endosomal positioning of SARS-CoV-2 spike receptor-binding domains 64]. In late endosome-early lysosome where the pH is acidic (pH 5.5-4.5), the spike adopts an all-RBD-down conformation, which provides a potential means of immune evasion from RBD-up-recognizing antibody [64]. Shed antibodies no longer bind the spike protein, which can now be activated by the low-pH protease TMPRSS2 [64]. TMPRSS2 then cleaves the spike protein which allows the SARS-CoV-2 virion to enter the cytosol.

### Tromethamine (TRIS)

In some embodiments, the composition of the present disclosure further comprises a buffer. The buffer adjusts the pH of the composition to desired values, such as pH between about 6 to about 9. In some embodiments, the pH of the composition is between about 5.99 to about 7.84. In some embodiments, the pH of the composition is between about 7.35 to about 7.45. In some embodiments, the buffer is tromethamine adjusting the pH to pH about 7.0.

Tromethamine [Tris(hydroxymethyl)aminomethane], 2-Amino-2-hydroxymethyl-propane-1,3-diol. (THAM or TRIS) is an FDA approved drug product. Tromethamine is indicated for the prevention and correction of metabolic acidosis [65] and is a useful way to manage excessively high pCO2 in respiratory acidosis [66]. Tromethamine an organic amine buffer, is a solid that is readily soluble in water (Hospira, Inc., FDA Package insert) [67]; Molecular Formula C4H11NO3; Molecular Weight 121.14 g/mol; pKa 7.82 @ 37° C.; CAS No. 77-86-1, and has the following structural formula:

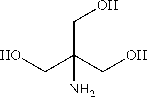

### Tromethamine CAS No. 77-86-1

Alkali therapy with sodium bicarbonate has specific side effects. It can lead to hypernatremia, hyperosmolality, and volume overload. CO2 may rise due to buffering of protons by bicarbonate (HCO3−+H+↔H2CO3↔H2O+CO2), and this may even lead to intracellular acidosis by diffusion of PaCO2 into the cytoplasm [68,69,70,71]. TRIS, a weak base with a pKa of 7.8 has been proposed as an alternative alkalinizing agent [68,72,73,74]. TRIS exerts its buffering capabilities by binding both carbon dioxide and metabolic acids through the following mechanisms [68,74]:

(R—NH2=unprotonated TRIS, and R—NH3+=protonated TRIS)

R—NH2+H2O+CO2↔R—NH3++HCO3 [symbol] R—NH2+H+↔R—NH3+

Tromethamine acts as a proton acceptor and prevents or corrects acidosis by actively binding hydrogen ions (H+) [67]. It binds not only cations of fixed or metabolic acids, but also hydrogen ions of carbonic acid, thus increasing bicarbonate anion (HCO3−) [67].

To alkalize the blood, patients must be able to eliminate CO2 by increasing their minute ventilation [74]. This is often not realistic in patients with acute circulatory shock and/or respiratory failure [74]. The resultant build-up of CO2 drives the equation to the left and creates more acid [74]. CO2 readily diffuses across cell membranes and generation of more CO2 may in fact create intracellular acidosis [74,75].

TRIS is thought to exert effects in both the extracellular space and intracellular space [74,72]. TRIS has a greater buffering capacity than bicarbonate (pKa of 7.82 versus 6.1, respectively) [73], and is effective in buffering both metabolic and respiratory acidosis [76]. In respiratory acidosis TRIS lowers CO2 while producing bicarbonate [77]. Unlike sodium bicarbonate, which requires patients to augment their alveolar minute ventilation to eliminate CO2, TRIS forms (R—NH3+) which is renally excreted by glomerular filtration [74]. The protonated buffer gets eliminated in the urine, which results in TRIS being effective in a closed system independent of pulmonary function [74].

The closed system buffering capabilities of TRIS makes it highly useful in disease processes such as ARDS [78], where CO2 elimination is hindered by permissive hypercapnea strategies employed to prevent further lung injury and increased dead space minute ventilation [74].

As sodium bicarbonate leads to an increase of serum sodium and TRIS to a decrease, TRIS may be the alkalinizing agent of choice in patients with hypernatremia [68]. Similarly, because sodium bicarbonate increases PaCO2 and TRIS may even decrease PaCO2, sodium bicarbonate is contraindicated and TRIS preferred in patients with mixed acidosis with high PaCO2 levels [68].

Respiratory acidosis is commonly present in patients with respiratory failure. The usual treatment of hypercapnia is to increase ventilation [79]. During the recent surge of COVID-19, respiratory acidosis unresponsive to increased mechanical ventilatory support was common. Increasing mechanical ventilation comes at the expense of barotrauma and hemodynamic compromise from increasing positive end-expiratory pressures or minute ventilation [79].

Respiratory acidosis occurs frequently in patients with COVID-19, probably because of increased dead space as the result of microthrombotic obstruction as well as by blood flow through poorly (low V/Q) and non-ventilated (shunt) regions [79].

Administering sodium bicarbonate for respiratory acidosis that cannot be improved by increasing mechanical ventilation is not an evidence-based strategy. [79]. There is a lack of clinical evidence that administration of sodium bicarbonate for respiratory acidosis has a net benefit [79]. Likewise, although severe metabolic acidosis is associated with increased mortality, administration of bicarbonate to reverse adverse consequences of acidemia has not been supported by clinical trials [79].

Another reason why TRIS is a more suitable buffer than sodium bicarbonate is that TRIS is used to increase the permeability of the cell membrane [80] without the assistance of integral transport proteins. TRIS is also a component of the Moderna COVID-19 vaccine [81]. HCO3− on the other hand, is a charged ion which makes it impermeable to the cell membrane. In order for HCO3− to traverse the cell membrane, its transport needs to be facilitated by integral membrane proteins. These bicarbonate transport proteins belong to the SLC4A and SLC26A families of bicarbonate transporters and move bicarbonate across the membrane [82,83,84]. Similarly, due to their ionic nature, acetate and phosphate transport across the cell membrane also need to be facilitated by integral transport proteins.

Yet another reason why TRIS is more suitable than NaHCO3 and other ionic buffers like phosphate and acetate is that TRIS is a non-ionic compound. The non-ionic nature of the TRIS buffer was paramount in reducing the osmolality of the formulation to less than 600 mOsm/kg. Potential vein damage can be a concern if the osmolality was greater than 600 mOsm/kg. [85]

Still another reason why TRIS is more suitable than NaHCO3 relates to the use of TRIS for the treatment of severe lactic acidosis. Marfo et al, demonstrated the use of Tris-hydroxymethylaminomethane in severe lactic acidosis due to highly active antiretroviral therapy (HAART). TRIS generates serum bicarbonate, and reduces the level of carbon dioxide in arterial blood. Both of these qualities appear to make TRIS an ideal agent for treating lactic acidosis caused by HAART [86].

Advantages of using a TRIS buffer solution in the disclosure versus a phosphate-buffered saline solution includes: phosphate tends to chelate with metal ions, whereas TRIS does not precipitate with divalent cations such as Ca+2, Mg+2, Fe+2, Zn+2 or heavy metal ions. As such, TRIS buffers are preferable over phosphate buffers to avoid complex formation with ionic species [87]; phosphate may inhibit enzymatic reactions [88], whereas TRIS is inert to many enzyme reactions; the effective buffering range of the TRIS buffer is between 7.0 and 9.2 [89], compared to the effective phosphate buffering range of 5.8 to 8.0 [88]; regarding vaccines, phosphate buffers are known to be suboptimal for freezing due to their propensity to precipitated and cause abrupt pH changes upon the onset of ice crystallization compared to TRIS buffers [90].

TRIS acts as a stabilizer and pH modifier in the intravenous formulation. In some embodiments, tromethamine utilized in the disclosure has an osmolality of greater than 260 mOsm/kg and less than 601 mOsm/kg in the reconstituted parenteral solution.

### Preparation of the Compounds and Compositions

Compounds of the present disclosure may be synthesized according to standard methods known in the art [see, e.g. Morrison and Boyd in “Organic Chemistry”, 6th edition, Prentice Hall (1992), the contents of which are herein incorporated by reference in their entirety]. Some compounds and/or intermediates of the present disclosure may be commercially available, known in the literature, or readily obtainable by those skilled in the art using standard procedures. Some compounds of the present disclosure may be synthesized using schemes, examples, or intermediates described herein. Where the synthesis of a compound, intermediate or variant thereof is not fully described, those skilled in the art can recognize that the reaction time, number of equivalents of reagents and/or temperature may be modified from reactions described herein to prepare compounds presented or intermediates or variants thereof and that different work-up and/or purification techniques may be necessary or desirable to prepare such compounds, intermediates, or variants.

Synthesized compounds may be validated for proper structure by standard methods well known to those skilled in the art, such as nuclear magnetic resonance (NMR) spectrometry, mass spectrometry, and/or infrared absorption spectroscopy.

As a non-limiting example, 1:1 (molar ratio) portion of Aspirin and L-Theanine are put in a mortar. The mixture is wetted with solvent (e.g., 70% aqueous isopropanol) and ground with pestle until the mixture is free powder. The mixture is then unloaded from the mortar. Tromethamine and zinc can be added to the cocrystal to obtain the composition of the present disclosure.

In some embodiments, the molar ratio of aspirin and L-theanine is 1:1. In some embodiments, the molar ratio of aspirin, L-theanine, and tromethamine is 1:1:z, wherein z is between about 1 and about 5. For example, z may be between about 1.0 and about 1.9, between about 2.0 and about 2.9, between about 3.0 and about 3.9, between about 4.0 and about 4.9.

In some embodiments, the weight percentage (wt. %) of aspirin cocrystal is between about 10% and about 50% (such as between about 10% and about 20%, between about 21% and about 30%, between about 31% and about 40%, between about 41% and about 50%), the wt. % of L-theanine cocrystal is between about 10% and about 50% (such as between about 10% and about 20%, between about 21% and about 30%, between about 31% and about 40%, between about 41% and about 50%), the wt. % of tromethamine is between about 10% and about 60% (such as between about 10% and about 20%, between about 21% and about 30%, between about 31% and about 40%, between about 41% and about 50%, between about 50% and about 60%).

In some embodiments, the cocrystal composition comprises between about 10 mg and about 1 g of aspirin cocrystal, between about 10 mg and about 1 g of L-theanine cocrystal, between about 100 mg to about 500 mg of tromethamine, and between about 0.1 mg and about 100 mg of zinc. In some embodiments, the cocrystal composition comprises between about 200 mg and about 500 mg of aspirin cocrystal, between about 200 mg and about 500 mg of L-theanine cocrystal, between about 150 mg to about 250 mg of tromethamine, and between about 50 mg and 65 mg of zinc.

In some embodiments, the composition of the present disclosure can be prepared by mixing the components with water to obtain a mixture, and then diluting the mixture with water to obtain the composition. The Tyndall effect of the composition is monitored by any known method in the art, such as shining a laser beam through the solution. In some embodiments, the composition of the present disclosure exhibited a weak or no Tyndall effect.

### Methods of Use

Cocrystal compositions of a drug from a specified drug class, and the enantiomers, L- and D-isomers, D, L-racemic mixture, S- and R-isomers, S, R-racemic mixtures, all rotamers, tautomers, salt forms, and hydrates of the alpha and beta amino acids of theanine in which the N-substituted functional R1-group [C4 or gamma-CH2-C(O)—NR1] may contain linear, cyclic, or branched alkyl groups and derivatives thereof; linear, cyclic or branched alkenyl groups and derivatives thereof; and aromatic radicals (which may be aryl radicals) and derivatives thereof making up all the analogue forms of theanine.

In one embodiment, the disclosure relates to a method of treating a disease or disorder in a subject in need thereof, comprising administering to the subject a dosage form of the compositions of the disclosure.

In some embodiments, zinc utilized in the disclosure is in the form of zinc chloride (ZnCl2).

In some embodiments, compositions comprising water soluble aspirin and L-theanine, together with tromethamine are administered via intravenous infusion.

In some embodiments, the water-soluble aspirin/L-theanine cocrystal, tromethamine formulation reduces the formation of pro-inflammatory cytokines in COVID-19 disease.

In some embodiments, zinc has a synergistic effect with the water-soluble aspirin/L-theanine cocrystal, tromethamine formulation in the treatment of the intense inflammatory response related to cytokine storm in COVID-19 disease.

In some embodiments, zinc has a synergistic immuno-modulatory effect with the water-soluble aspirin/L-theanine cocrystal, tromethamine formulation to target interleukin-6 in the treatment of cytokine storm in COVID-19 disease.

In some embodiments, zinc has a synergistic anti-viral effect with the water-soluble aspirin/L-theanine cocrystal, tromethamine formulation to target interleukin-6 in the treatment of cytokine storm in COVID-19 disease.

Accordingly, it is an object of the disclosure to provide a composition comprising cocrystals of aspirin and L-theanine, with tromethamine to downregulate IL-6 in the treatment of cytokine storm in COVID-19 disease.

In one embodiment, compositions comprising cocrystals of aspirin and L-theanine with tromethamine are used to treat an acute inflammatory disease associated with COVID-19 disease in subjects in need thereof. The subjects may also receive zinc or dipyridamole treatments.

In one embodiment, compositions comprising cocrystals of aspirin and L-theanine with tromethamine are used to treat a chronic inflammatory disease associated with COVID-19 disease in subjects in need thereof. The subjects may also receive zinc or dipyridamole treatments.

In one embodiment, compositions comprising cocrystals of aspirin and L-theanine with tromethamine are used for downregulating interleukin-6 in the treatment of cytokine storm in COVID-19 disease in subjects in need thereof. The subjects may also receive zinc or dipyridamole treatments.

In one embodiment, compositions comprising cocrystals of aspirin and L-theanine with tromethamine are used for treating the intense inflammatory reaction associated with COVID-19 disease in subjects in need thereof. The subjects may also receive zinc or dipyridamole treatments.

In one embodiment, compositions comprising cocrystals of aspirin and L-theanine with tromethamine are used for treating the intense inflammatory reaction, where the inflammatory reaction is a cytokine storm consisting of dysregulated immune responses orchestrated by inflammatory cytokines, lymphocyte cell death, hypoxia, and endothelial damage associated with COVID-19 disease in subjects in need thereof. The subjects may also receive zinc or dipyridamole treatments.

In some embodiments, compositions comprising water soluble cocrystals of aspirin and L-theanine, together with tromethamine administered via the intravenous route inhibits IL-6 synthesis through immuno-modulatory cyclo-oxygenase 2 (COX-2) inhibition of prostaglandin E2 and nuclear factor-kappa B antagonism for treating the intense inflammatory reaction, where the inflammatory reaction is a cytokine storm consisting of dysregulated immune responses orchestrated by inflammatory cytokines, lymphocyte cell death, hypoxia, and endothelial damage associated with COVID-19 disease in subjects in need thereof. The subjects may also receive zinc or dipyridamole treatments.

In some embodiments, compositions comprising water soluble cocrystals of aspirin and L-theanine, together with tromethamine administered via intravenous infusion inhibits viral replication due to its inhibitory effect on PGE2 and its effect on Type I Interferon Alpha (IFN-α) by inducing the upregulation of numerous genes that activate both innate and adaptive immunity for rapidly controlling viral replication in subjects in need thereof. The subjects may also receive zinc or dipyridamole treatments.

In some embodiments, compositions comprising water soluble cocrystals of aspirin and L-theanine, together with tromethamine administered via intravenous infusion inhibits the activity of IκB kinase-β, thereby preventing activation of nuclear factor-KB, which is involved in the pathogenesis of inflammation in subjects in need thereof. The subjects may also receive zinc or dipyridamole treatments.

In some embodiments, compositions comprising water soluble cocrystals of aspirin and L-theanine, together with tromethamine and zinc administered via intravenous infusion triggers anti-inflammatory 15-epi-lipoxin A4 and induction of apoptosis of inflammatory cells via the mitogen-activated protein kinase pathway in subjects in need thereof. The subjects may also receive zinc or dipyridamole treatments.

In some embodiments, compositions comprising water soluble cocrystals of aspirin and L-theanine, together with tromethamine administered via intravenous infusion reduces the accumulation of inflammatory cells in an adenosine-dependent manner in subjects in need thereof. The subjects may also receive zinc or dipyridamole treatments.

In some embodiments, compositions comprising water soluble cocrystals of aspirin and L-theanine, together with tromethamine administered via intravenous infusion inhibits IL-6 expression of metallothioneins and α2-macroglobulin by modulation of the pro-inflammatory response by targeting nuclear factor kappa B(NF-κB), a transcription factor that is the master regulator of proinflammatory responses for the treatment of COVID-19 cytokine storm, in subjects in need thereof. The subjects may also receive zinc or dipyridamole treatments.

In some embodiments, derivatives prepared using water soluble cocrystals of aspirin and L-theanine, together with tromethamine administered via intravenous infusion inhibits IL-6 mediated activation of STAT3 for the treatment of COVID-19 cytokine storm in subjects in need thereof. The subjects may also receive zinc or dipyridamole treatments.

In some embodiments, compositions comprising water soluble cocrystals of aspirin and L-theanine, together with tromethamine administered via intravenous infusion has antioxidant properties neutralizing free radicals for the treatment of COVID-19 cytokine storm in subjects in need thereof. The subjects may also receive zinc or dipyridamole treatments.

In one embodiment, compositions comprising water soluble cocrystals of aspirin and L-theanine, together with tromethamine are used to treat an endotheliopathy associated with COVID-19 disease in subjects in need thereof. The subjects may also receive zinc or dipyridamole treatments.

In one embodiment, compositions comprising water soluble cocrystals of aspirin and L-theanine, together with tromethamine are used to treat a coagulopathy associated with COVID-19 disease, where the coagulopathy is a hypercoagulable state resulting in an endotheliopathy in subjects in need thereof. The subjects may also receive zinc or dipyridamole treatments.

In one embodiment, compositions comprising water soluble cocrystals of aspirin and L-theanine, together with tromethamine are used to treat a coagulopathy associated with COVID-19 disease, where the coagulopathy is a hypercoagulable state resulting in an endotheliopathy that involves the arteries, arterioles, veins, venules, and capillaries, in subjects in need thereof. The subjects may also receive zinc or dipyridamole treatments.

In one embodiment, compositions comprising water soluble cocrystals of aspirin and L-theanine, together with tromethamine are used to treat a coagulopathy associated with COVID-19 disease, where the coagulopathy is a hypercoagulable state resulting in an endotheliopathy wherein the endotheliopathy results in a prothrombotic state, in subjects in need thereof. The subjects may also receive zinc or dipyridamole treatments.

In one embodiment, compositions comprising water soluble cocrystals of aspirin and L-theanine, together with tromethamine are used to treat a coagulopathy associated with COVID-19 disease, where the coagulopathy is a hypercoagulable state resulting in an endotheliopathy wherein the endotheliopathy consists of, but not limited to inflammatory injury, in subjects in need thereof. The subjects may also receive zinc or dipyridamole treatments.

In one embodiment, compositions comprising water soluble cocrystals of aspirin and L-theanine, together with tromethamine are used to treat a coagulopathy associated with COVID-19 disease, where the coagulopathy is a hypercoagulable state resulting in an endotheliopathy wherein the endotheliopathy, consists of, but not limited to endothelial damage, in subjects in need thereof. The subjects may also receive zinc or dipyridamole treatments.

In one embodiment, compositions comprising water soluble cocrystals of aspirin and L-theanine, together with tromethamine are used to treat a coagulopathy associated with COVID-19 disease, where the coagulopathy is a hypercoagulable state resulting in an endotheliopathy wherein the endotheliopathy consists of, but not limited to vascular leakage, in subjects in need thereof. The subjects may also receive zinc or dipyridamole treatments.

In one embodiment, compositions comprising water soluble cocrystals of aspirin and L-theanine, together with tromethamine are used to treat a coagulopathy associated with COVID-19 disease, where the coagulopathy is a hypercoagulable state resulting in an endotheliopathy wherein the endotheliopathy results in microvascular thrombosis, in subjects in need thereof. The subjects may also receive zinc or dipyridamole treatments.

In one embodiment, compositions comprising water soluble cocrystals of aspirin and L-theanine, together with tromethamine are used to treat a coagulopathy associated with COVID-19 disease, where the coagulopathy is a hypercoagulable state resulting in an endotheliopathy wherein the endotheliopathy results in microvascular thrombosis, specifically in the alveoli epithelium of the lungs, in subjects in need thereof. The subjects may also receive zinc or dipyridamole treatments.

In some embodiments, compositions comprising water soluble cocrystals of aspirin and L-theanine, together with tromethamine are used to treat a coagulopathy associated with COVID-19 disease, where the coagulopathy is a hypercoagulable state associated with an antiphospholipid syndrome, a potentially life-threatening thrombophilia in which patients develop pathogenic autoantibodies targeting phospholipids and phospholipid-binding proteins (aPL antibodies), in subjects in need thereof. The subjects may also receive zinc or dipyridamole treatments.

In some embodiments, compositions comprising water soluble cocrystals of aspirin and L-theanine, together with tromethamine are used to treat a coagulopathy associated with COVID-19 disease, where the coagulopathy is a hypercoagulable state associated with the aPL antibodies anticardiolipin IgG, IgM, and IgA; anti-β2 glycoprotein I IgG, IgM, and IgA; and anti phosphatidylserine/prothrombin (aPS/PT) IgG and IgM, in subjects in need thereof. The subjects may also receive zinc or dipyridamole treatments.

In some embodiments, compositions comprising water soluble cocrystals of aspirin and L-theanine, together with tromethamine are used to treat a coagulopathy associated with COVID-19 disease, where the coagulopathy is a hypercoagulable state and the disclosure is expected to reduce the production of any potential autoantibodies that may cause clots in the arteries, veins, and capillaries, in subjects in need thereof. The subjects may also receive zinc or dipyridamole treatments.

In some embodiments, compositions comprising water soluble cocrystals of aspirin and L-theanine, together with tromethamine are used to treat a coagulopathy associated with COVID-19 disease, where the coagulopathy is a hypercoagulable state and the disclosure is expected to reduce the production of any potential antiphospholipid antibodies that may cause clots in the arteries, veins, and capillaries, in subjects in need thereof. The subjects may also receive zinc or dipyridamole treatments.

In some embodiments, compositions comprising water soluble cocrystals of aspirin and L-theanine, together with tromethamine are used to treat a coagulopathy associated with COVID-19 disease, where the coagulopathy is a hypercoagulable state and the SARS-CoV-2 is an Alpha, Beta, Gamma, Delta, Epsilon, Eta, Iota, Kappa, Lambda, Mu, Theta, Zeta, R.1 variant, THU variant (B.1.640.2), omicron Pango lineage B.1.1.529, omicron descendent Pango lineages BA.1, BA.1.1, BA.2, BA.3, BA.4, BA.5, or any variants, including hybrid variants, or sub-variants thereof, or any mutations, specifically the D614G mutation in the Spike protein, the P323L mutation in the NSP12 polymerase, and the C241U noncoding mutation in the 5-end, or any mutations showing an atypical combination, in subjects in need thereof. The subjects may also receive zinc or dipyridamole treatments.

In some embodiments, compositions comprising water soluble cocrystals of aspirin and L-theanine, together with tromethamine are used to treat a coagulopathy associated with COVID-19 disease, where the coagulopathy is a hypercoagulable state and the SARS-CoV-2 variants include all of their associated lineages and sub-lineages according to the Pango Nomenclature System, in subjects in need thereof. The subjects may also receive zinc or dipyridamole treatments.

### Dipyridamole (Persantine)

Dipyridamole [2,6 bis-(diethanolamino)-4,8 dipiperidino-pyrimido-(5,4-d) pyrimidine] is a platelet aggregation inhibitor that inhibits the activity of adenosine deaminase and phosphodiesterase, resulting in accumulation of adenosine, adenine nucleotides, and cyclic AMP mediators which inhibit platelet aggregation and cause vasodilation [91].

Dipyridamole acts as a phosphodiesterase inhibitor resulting in reduced platelet aggregation and anticoagulation. Liu, X., et al demonstrated the therapeutic effect of dipyridamole on COVID-19 patients with coagulation dysfunction [92].

Dipyridamole has a broad spectrum antiviral activity, particularly efficacious against the positive-stranded RNA viruses [93,94]. Second, it suppresses inflammation and promotes mucosal healing [93,95]. Third, as a pan-PDE inhibitor, dipyridamole may prevent acute injury and progressive fibrosis of the lung, heart, liver, and kidney [93,96].

In addition to being a platelet inhibitor, dipyridamole has the additional effect of lowering pulmonary hypertension without significantly affecting systemic blood pressure [97].

In some embodiments, dipyridamole increases myocardial perfusion [98].

In some embodiments, dipyridamole inhibits the formation of pro-inflammatory cytokines and together with ASA protects against endothelial damage [98].

Dipyridamole has recently been shown to suppress SAR-CoV-2 replication in vitro [93]. A recent proof-of-concept trial involving 31 patients with COVID-19, showed that dipyridamole significantly decreased D-Dimer (P>0.05), increased lymphocyte and plasma platelet recovery, and markedly improved clinical outcomes in comparison to the controlled patients [99]. Liu, X et al showed that dipyridamole bound to the SARS-CoV-2 protease Mpro after identified via the virtual screening and bioassay validation, suppressed viral replication in vitro [93].

ASA is a well-established anti-inflammatory that may contain the ideal pharmacological properties when administered intravenously to target the cycle of pulmonary damage in patients with COVID-19 disease, without compromising adaptive immunity and viral clearance. Oral ASA is largely ineffective due to de-acetylation in the gastric mucosa (a major cause of gastric toxicity) and decreased bioavailability of the active drug (acetyl form) to the respiratory endothelium).

IV ASA is readily available for repurposing. Combining acetylsalicylic acid and dipyridamole (an antiplatelet vasodilator) has additional mechanistic synergy, and at rapid high doses can directly counter cardio-respiratory vasoconstriction due to hypoxia (as seen by its' use as a cardio-vasodilator in the Dipyridamole stress test) without significantly compromising hemodynamic stability in hypotensive patients [97].

In some embodiments, compositions of the disclosure comprising intravenous cocrystals of aspirin and L-theanine are given to subjects in need thereof. The subjects may also receive sublingual dipyridamole suspension, which has anticoagulant, anti-inflammatory, antiviral, and immunomodulatory properties that are synergistic for the treatment of COVID-19 coagulopathy.

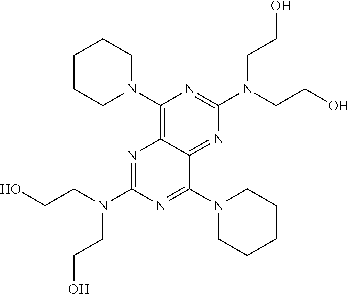 [SMILES: OCCN(CCO)c1nc(N2CCCCC2)c2nc(N(CCO)CCO)nc(N3CCCCC3)c2n1]

### Dipyridamole Chemical Structure Case No. 58-32-2

In some embodiments, the amount of dipyridamole given to a subject is between 25 mg and 600 mg. In some embodiments, dipyridamole is administered via oral or sublingual routes. In some embodiments, dipyridamole is in the form of oral solids (tablet, oral disintegrating tablet), oral liquids, clear homogeneous solutions, suspension or powder.

### Zinc

As used herein, zinc refers to any compound that has zinc in it. Non-limiting examples of zinc include zinc chloride (ZnCl2), zinc sulfate (ZnSO4), zinc nitrate (Zn(NO3)2), or a hydrate or salt thereof.

Zinc is an essential trace element that plays a role in the body's immune system. Zinc chloride (ZnCl2), CAS number 7646-85-7 and Molecular weight 136.30, is hygroscopic. Zinc chlorides are highly soluble in water. The water solubility of zinc chloride is 432 g/100 ml water at 25° C. [100].

Zinc is involved in the modulation of the proinflammatory response by targeting Nuclear Factor Kappa B (NF-κB), a transcription factor that is the master regulator of pro-inflammatory responses [101]. Metallothioneins (MTs) are cysteine-rich 6-7 kDa proteins that bind metal ions such as zinc [101,102]. α2-macroglobulin (A2M) is another zinc-binding protein which is an inhibitor of matrix metalloproteases (MMPs) and it is required to remove proteolytic potential, when MMPs increase, forming A2M-proteinase complexes. It has a very high affinity to zinc, where zinc is required for the activation of A2M and also for the binding of A2M with cytokines [101,103]. The cytokine interleukin 6 (IL6) induces the expression of MT and A2M and consequently reduces zinc availability [101]. IL-6 is released during the acute phase of an inflammatory response. Zinc deficiency increases the production of pro-inflammatory cytokines, such as interleukins IL-1β, IL-6, and tumor necrosis factor (TNF)-α [101].

Proper modulation of inflammatory pathways is required to achieve adequate response to various stimuli such as stress, free radicals, cytokines, or bacterial and viral antigens. One of the main inflammatory pathways is the nuclear factor kappa-light-chain-enhancer of activated B cells (NF-κB) signaling pathway. It regulates the genes controlling apoptosis, cell adhesion, proliferation, tissue remodeling, the innate and adaptive immune responses, inflammatory processes, and cellular-stress responses. Subsequently, it influences the expression of pro-inflammatory cytokines such as TNF-α, IL-1β, IL-6, IL-8, and MCP (monocyte chemoattractant protein)-1. NF-κB is one of the most versatile regulators of gene expression [101,104].

In some embodiments, the amount of zinc in the intravenous dosage form is between 0.1 mg and 100 mg. In some embodiments, the wt. % of zinc in the intravenous cocrystal formulation is between about 1% to about 10%.

### Pro-Inflammatory Signaling Pathway Influenced by Zinc

Similar to TLR signaling, IL-1, and TNF-R signaling pathways converge on a common IκB kinase complex that phosphorylates the NF-κB inhibitory protein, resulting in the release of NF-κB and its translocation to the nucleus. Zinc prevents the dissociation of NF-κB from its corresponding inhibitory protein, thus preventing the nuclear translocation of NF-κB and inhibiting subsequent inflammation. Zinc also inhibits IL-6-mediated activation of STAT3 (Signal Transducer and Activator of Transcription 3). Zinc acts as anti-inflammatory element influencing major pro-inflammatory signaling pathways [101].

### Anti-Inflammatory Signaling Pathways Influenced by Free Zinc

TGFβ signaling is dependent on a dynamic on and off switch in Smad activity. Free zinc is a cofactor in Smad proteins and promote Smad 2/3 nuclear translocation and transcriptional activity. Zinc regulates IL-2 signaling pathway via blocking MAP kinase phosphatase (MKP) in extracellular signal-regulated kinases (ERK) ½ pathways and Phosphatase and tensin homologue (PTEN) which opposes phosphoinositide 3-kinase (PI3K) function in PI3k/Akt pathway. Free zinc phosphorylates STATE and promotes translocation of STAT dimers into the nucleus, hence promote the anti-inflammatory effects of IL-4 [101].

When inflammation starts, NF-κB directly activates the expression of ZIP8, which then localizes to the plasma membrane, thereby mediating Zn uptake. When zinc enters the cytosol via ZIP8 it goes on to inhibit IKKβ kinase activity, which leads to the attenuation of the pro-inflammatory response [101,105].

### Antioxidant Effects of Zinc

Zinc has several antioxidant effects [101]. Under non-pathological conditions, cells produce ROS during cellular respiration. However, excessive production of ROS and the decreased rate of its neutralization and removal by antioxidant defense mechanisms lead to an imbalance between oxidants and antioxidants which results in oxidative stress [101,106]. Plasma zinc concentrations rapidly decline during acute phase response to different stimuli such as stress, infection, and trauma. Consequently, zinc is shuttled into cellular compartments, where it is utilized for protein synthesis, neutralization of free radicals, and to prevent microbial invasion. This redistribution of zinc during inflammatory events seems to be mediated by cytokines. Several studies have demonstrated how patients with acute illnesses present with hypozincemia along with elevated cytokine production [101,107,108].

In SARS-CoV-2, respiratory compromise results in impaired oxygenation and hypoxia to various end organs. Such hypoxia may contribute to end-organ failure and increase the risk of mortality. Specifically, such COVID-19 associated hypoxia has been proposed to be contributory to cardiac injury [109,110], hepatic injury [109,111], and renal injury [109,112,113].

Hypoxia and oxidative stress, result in an increase in reactive oxygen species (ROS), including superoxide (O2−), hydrogen peroxide (H2O2) and hydroxyl radical (.OH)—which result in intracellular damage [109,114,115]. Zinc appears to limit ROS production by several mechanisms. First, MTs, small cysteine-rich and heavy metal-binding proteins, participate in the intracellular defense against reactive oxygen and nitrogen species [109,116], and zinc has been shown to induce MT mRNA and protein expression. Second, zinc competes with Fe2+ and Cu2+ ions for binding to cell membranes and proteins—normally, these active metals catalyse the production of hydroxyl radical from H2O2 via Fenton chemistry. Third, zinc upregulates the production and activation of antioxidant proteins, molecules and enzymes such as glutathione, catalase and superoxide dismutase, which catalyse O2− to oxygen or H2O2 [109,117]. Finally, zinc reduces the activation of oxidant-promoting enzymes such as inducible nitric acid synthase and NADPH enzyme, which catalyse oxygen to O2−. Zinc may provide protection against the hypoxic injury that critically ill patients with COVID-19 may experience.

### Effect of Zinc on Proteolytic Processing of Replicase Polyproteins and the RNA-Dependent RNA Polymerase (RdRp) Activity

In coronaviruses, zinc inhibits both the proteolytic processing of replicase polyproteins and the RNA-dependent RNA polymerase (RdRp) activity [109,118]. DNA and RNA polymerases use divalent metal ions like Mg2+ as a cofactor, and one possible mechanism is that zinc displaces Mg2+ and subsequently inhibits RdRp activity [109,119]. In support is the observation that various divalent metals ions sustained the activity of polio-virus RdRp in the following preference Mn2+>Co2+>Ni2+>Fe2+>Mg2+>Ca2+>Cu2+ [109,120]. In contrast, zinc was incapable of sustaining RdRp catalysed nucleotide incorporation [109,120]. A zinc-binding pocket has been identified in the Dengue virus and SARS-coronavirus RdRp [109,118]. Therefore, it is possible that binding of zinc may induce a structural change in the conformation of RdRp which disables RdRp to catalyse nucleotide incorporation. Adding high concentrations of zinc ions to cells impairs viral polyprotein processing which is integral to virus replication [109,121].

### Safety of High Dose Intravenous Zinc (HDIZn)

The safety of HDIVZn has been addressed in previous literature [109,122,123,124,125]. Elemental zinc has been administered at a substantially higher dose (ranging from 26.4 to 37.5 mg/d for 8 successive days) in the treatment of burns and did not produce any side effects in humans [109,122,123,124]. A published phase I clinical trial in critically ill children with suspected zinc deficiency administered zinc intravenously at an elemental dose of 0.75 mg/kg/d for 7 days without producing any adverse effects [126,125].

In some embodiments, zinc is provided in the form of zinc chloride. In some embodiments, zinc chloride is administered as an aqueous solution that can be administered before or after the compositions comprising aspirin and L-theanine co-crystals together with tromethamine between about 5 mg to about 50 mg of zinc chloride may be administered.

In one embodiment, zinc chloride is administered as an aqueous solution that can be administered before or after the compositions comprising aspirin and L-theanine co-crystals together with tromethamine. Between about 0.5 mg to about 50 mg of zinc chloride may be administered. As a non-limiting example, the zinc chloride injection contains zinc chloride 10.6 mg in 2 mL water for injections. Each 2 mL contains 0.078 mmol (0.156 mEq) zinc and 0.156 mmol (0.156 mEq) of chloride. The zinc chloride is administered as follows: intravenous infusion by diluting each 2 mL vial in 1 liter solution (5% glucose or NaCl 0.9%) administered over 8 to 24 hours (along with the Aspirin L-theanine cocrystal and tromethamine).

In one embodiment, compositions of aspirin and L-theanine co-crystals together with tromethamine and zinc have a synergistic effect in the treatment of the intense inflammatory response related to cytokine storm in COVID-19 disease.

In some embodiments, a method of treating acidosis in a subject in need thereof is provided, wherein the method comprises administering an effective amount of the composition comprising the cocrystals of aspirin and L-theanine, wherein the subject has COVID-19. The composition may further comprise tromethamine. The method may further comprise administering zinc via intravenous infusion. The acidosis may be respiratory acidosis as a result of COVID-19, metabolic acidosis as a result of COVID-19, lactic acidosis as a result of COVID-19, mixed respiratory acidosis and metabolic acidosis, or mixed respiratory acidosis and lactic acidosis as a result of COVID-19.

In some embodiments, a method of preventing viral entry of a virus into the cytosol of a subject in need thereof is provided, wherein the method comprises administering an effective amount of the composition comprising the cocrystals of aspirin and L-theanine, wherein the subject has COVID-19. The composition may further comprise tromethamine. The method may further comprise administering zinc via intravenous infusion. The pH of the acidic endo-lysosomes is increased to a physiologic pH or basic pH. In one embodiment, the subject may have respiratory acidosis, metabolic acidosis, lactic acidosis or a combination thereof. In one embodiment, the subject has coagulopathy.

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

The present invention is further illustrated by the following non-limiting examples.

### EXAMPLES

### Example 1. Reconstitution Studies

### (I) Preparation of Tromethamine (TRIS) Buffer Solutions

A series of IRIS buffer solutions were prepared by first weighing out an appropriate amount of solid tromethamine (formula molecular weight=121.14 g/mol), and then dissolving this in 10 mL of water.

### (II) Preparation of Reconstitution Solutions

Approximately 100-mg of aspirin-theanine cocrystal was weighted in a 25-mL beaker, and then adding the 10-mL of IRIS buffer whose concentration had been pre-determined as a result of the buffer preparation.

### (III) Measurements

The solution pH (in this work, using a model 630 Fisher Accumet pH meter, equipped with a glass combination electrode) was measured, and then the osmolality of the solution (in this work, using a model 2430 Precision Systemsosmometer) was measured.

### Results

Using the procedure described above, a series of five reconstitution solutions were prepared and characterized. The compositions and resulting measured parameters are summarized in the following table:

| | Molarity of | | | |
|---|---|---|---|---|
| Milli- | theresulting | Weight | | |
| grams | TRIS- | ratio | | Measured |
| of TRIS | Solution | of TRIS/ | Measured | Osmolality |
| Taken | (mol/L) | Cocrystal | pH | (mosm/kg) |
| 175 | 0.144 | 1.75 | 5.99 | 527 |
| 152 | 0.125 | 1.52 | 6.51 | 504 |
| 199 | 0.164 | 1.99 | 7.03 | 551 |
| 216 | 0.178 | 2.16 | 7.48 | 568 |
| 248 | 0.205 | 2.48 | 7.84 | 600 |

### Example 2. Formulation Examples Using the Aspirin/L-Theanine Cocrystal Containing Added TRIS and Zinc

Target Formulation:

(1) 500 mg aspirin/theanine cocrystal: Equivalent to 254 mg aspirin (1.409 mmol) and 246 mg theanine (1.413 mmol)

### (2) TRIS 199 mg

(3) 0.194 mmol of a zinc salt: Using zinc nitrate (Zn(NO3)2) hexahydrate (Zn(NO₃)₂.6H₂O) as the source, 57.8 mg

Procedure:

(1) Dry ingredients were weighed out in a 50-mL beaker. 20 mL of water was added. The mixture was stirred until effervescence ceased, and then observations of apparent solution clarity were recorded. Using a handheld laser, the solution was interrogated for the presence of a Tyndall effect to evaluate the apparent degree of dissolution.
(2) Subsequently, the solution from step (1) was diluted to 1 liter with water, and then observations were recorded as for step (1).
(3) The laser wavelength span used in the experiments was 630-670 nanometers.

| | | | | Zinc (Zn | |
|---|---|---|---|---|---|
| | | L- | | (NO₃)₂• | |
| | Aspirin | theanine | TRIS | 6H₂0) | Total |
| Weight (mg) | 254 | 246 | 199 | 57.8 | 756.8 |
| Number of | 1.409 | 1.413 | 1.643 | 0.194 | 4.659 |
| Moles (mmol) | | | | | |
| Molar Ratio | 1.00 | 1.00 | 1.17 | 0.14 | / |
| Weight Ratio | 0.508 | 0.492 | 0.398 | 0.116 | / |
| (v.s. 500 | | | | | |
| mg cocrystal) | | | | | |
| Weight | 33.6 | 32.5 | 26.3 | 7.6 | 100 |
| percentage (%) | | | | | |

### Example 3. Formulation Examples Using the Aspirin/Theanine Cocrystal Containing Added Bicarbonate and Zinc

Target Formulation:

(1) 500 mg aspirin/theanine cocrystal: Equivalent to 254 mg aspirin (1.409 mmol) and 246 mg theanine (1.413 mmol)
(2) 0.194 mmol of a zinc salt: Using zinc nitrate hexahydrate as the source, 57.8 mg

### General Procedure:

Weigh out dry ingredients in a 50-mL beaker, and add 20 mL of water. Stir until effervescence ceases, and then record observations of apparent solution clarity. Using a handheld laser, interrogate the solution for the presence of a Tyndall effect to evaluate the apparent degree of dissolution.

Subsequently dilute the solution from step (1) to 1 liter with water, and then record observations as for step (1).

### Solution-1

0.512 g of aspirin/theanine cocrystal was weighed, corresponding to 260 mg of aspirin (1.443 mmol) and 252 mg of theanine (1.447 mmol). Then 0.124 g of sodium bicarbonate (1.476 mmol) was weighed in, for a bicarbonate/aspirin mole ratio of 1.02. After that, 58 mg of Zn(NO₃)₂.6H₂O (0.195 mmol) was weighed in.

20 mL of water was added to the weighed solids, and stirred until all effervescence had ceased. It was observed that complete dissolution of the solids did not take place, and the solution exhibited a strong Tyndall effect.

The solution was subsequently diluted to 1 liter with water, and after mixing it was observed that while the solution looked clear to the eye, it did show a definite Tyndall effect.

### Solution-2

0.504 g of aspirin/theanine cocrystal was weighed, corresponding to 256 mg of aspirin (1.420 mmol) and 248 mg of theanine (1.425 mmol). Then 0.258 g of sodium bicarbonate (3.071 mmol) was weighed in, for a bicarbonate/aspirin mole ratio of 2.16. After that, 61 mg of Zn(NO₃)₂.6H₂O (0.205 mmol) was weighed in.

20 mL of water was added to the weighed solids, and stirred until all effervescence had ceased. It was observed that the solution was visually clear, but that it did show a weak Tyndall effect.

Subsequently the solution was diluted to 1 liter with water, and after mixing it was observed that the solution was visually clear to the eye, while showing a weak Tyndall effect.

### Solution-3

0.521 g of aspirin/theanine cocrystal was weighed, corresponding to 264 mg of aspirin (1.468 mmol) and 257 mg of theanine (1.473 mmol). Then 0.366 g of sodium bicarbonate (4.357 mmol) was weighed in, for a bicarbonate/aspirin mole ratio of 4.36. After that, weighed in 55 mg of Zn(NO₃)₂.6H₂O (0.185 mmol).

20 mL of water was added to the weighed solids, and stirred until all effervescence had ceased. It was observed that the solution was visually clear, but that it did show a weak Tyndall effect.

Subsequently the solution was diluted to 1 liter with water, and after mixing it was observed that while the solution was visually clear to the eye, it did show a very weak Tyndall effect.

### Solution-4

0.517 g of aspirin/theanine cocrystal was weighed, corresponding to 262 mg of aspirin (1.457 mmol) and 255 mg of theanine (1.461 mmol). Then 0.468 g of sodium bicarbonate (5.571 mmol) was weighed in, for a bicarbonate/aspirin mole ratio of 3.82. After that, weighed in 58 mg of Zn(NO₃)₂.6H₂O (0.195 mmol).

20 mL of water was added to the weighed solids, and stirred until all effervescence had ceased. It was observed that while the solution was visually clear, it did show a very weak Tyndall effect.

Subsequently the solution was diluted to 1 liter with water, and after mixing it was observed that the solution was visually clear, and did not show a Tyndall effect.

### Solution-5

0.509 g of aspirin/theanine cocrystal was weighed, corresponding to 258 mg of aspirin (1.434 mmol) and 251 mg of theanine (1.439 mmol). Then 0.606 g of sodium bicarbonate (7.213 mmol) was weighed in, for a bicarbonate/aspirin mole ratio of 5.03. After that, 62 mg of Zn(NO₃)₂.6H₂O (0.208 mmol) was weighed in.

20 mL of water was added to the weighed solids, and stirred until all effervescence had ceased. It was observed that while the solution was visually clear, it did show a very weak Tyndall effect.

Subsequently the solution was diluted to 1 liter with water, and after mixing it was observed that the solution was visually clear, and did not show a Tyndall effect.

While a specific embodiment of the disclosure has been shown and described in detail to illustrate the application of the principles of the disclosure, it will be understood that the disclosure may be embodied otherwise without departing from such principles.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

### REFERENCES

1. Hirano, Toshio, and Masaaki Murakami. “COVID-19: A New Virus, but a Familiar Receptor and Cytokine Release Syndrome.” Immunity, vol. 52, no. 5, 2020, pp. 731-733.
2. Greinacher, A., Selleng, K., Mayerle, J., Palankar, R., Wesche, J., Reiche, S., Aebischer, A., Warkentin, T. E., Muenchhoff, M., Hellmuth, J. C., Keppler, O. T., Duerschmied, D., Lother, A., Rieg, S., Gawaz, M. P., Mueller, K. A. L., Scheer, C. S., Napp, M., Hahnenkamp, K., & Lucchese, G. (2021). Anti-platelet factor 4 antibodies causing VITT do not cross-react with SARS-CoV-2 spike protein. Blood, 138(14), 1269-1277.
3. Huang, Y., Yang, C., Xu, X., Xu, W., & Liu, S. (2020). Structural and functional properties of SARS-CoV-2 spike protein: potential antivirus drug development for COVID-19. Acta Pharmacologica Sinica, 41(9), 1141-1149.
4. Hoffmann, M., Kleine-Weber, H., Schroeder, S., Kruger, N., Herrler, T., Erichsen, S., Schiergens, T. S., Herrler, G., Wu, N.-H., Nitsche, A., et al. (2020). SARS-CoV-2 Cell Entry Depends on ACE2 and TMPRSS2 and Is Blocked by a Clinically Proven Protease Inhibitor. Cell 181, 271-280.
5. Iba, Toshiaki, et al. “Coagulopathy in COVID-19.” Journal of Thrombosis and Haemostasis, 2020.
6. To K F, Lo A W. Exploring the pathogenesis of severe acute respiratory syndrome (SARS): the tissue distribution of the coronavirus (SARS-CoV) and its putative receptor, angiotensin-converting enzyme 2 (ACE2). J Pathol. 2004; 203(3):740-743.
7. Guan W-J, Ni Z-Y, Hu Y, et al. Clinical characteristics of coronavirus disease 2019 in China. N Engl J Med. 2020; 382(18):1708-1720.
8. Wang D, Hu B, Hu C, et al. Clinical characteristics of 138 hospitalized patients with 2019 novel coronavirus-infected pneumonia in Wuhan, China. JAMA. 2020; 323(11):e201585.
9. Zhou, P., Yang, X. L., Wang, X. G., Hu, B., Zhang, L., Zhang, W., Si, H. R., Zhu, Y., Li, B., Huang, C. L., et al. (2020). A pneumonia outbreak associated with a new coronavirus of probable bat origin. Nature 579, 270-273.
10. Calabrese, C., Rajendram, P., Sacha, G. and Calabrese, L., Practical aspects of targeting IL-6 in COVID-19 disease. Cleveland Clinic Journal of Medicine, Oct. 6, 2020.
11. Pawar A, Desai R J, Solomon D H, et al. Risk of serious infections in tocilizumab versus other biologic drugs in patients with rheumatoid arthritis: a multidatabase cohort study. Ann Rheum Dis 2019; 78(4):456-464.
12. Monemi S, Berber E, Sarsour K, et al. Incidence of gastrointestinal perforations in patients with rheumatoid arthritis treated with tocilizumab from clinical trial, postmarketing, and real-world data sources. Rheumatol Ther 2016; 3(2):337-352.
13. Calabrese L H. Cytokine storm and the prospects for immunotherapy with COVID-19. Cleve Clin J Med 2020; 87(7):389-393.
14. Calabrese L H, Rose-John S. IL-6 biology: implications for clinical targeting in rheumatic disease. Nat Rev Rheumatol 2014; 10(12):720-727.
15. Vassilakopoulos, T., & Toumpanakis, D. (2007). Molecular mechanisms of action of Interleukin-6 (IL-6). PNEUMON, 20(2).
16. Busse W W, Lemanske R F, Jr. Asthma. N Engl J Med 2001; 344(5): 350-362. [symbol]
17. Moodley Y P, Scaffidi A K, Misso N L, et al. Fibroblasts isolated from normal lungs and those with idiopathic pulmonary fibrosis differ in interleukin-6/gp130-mediated cell signaling and proliferation. Am J Pathol 2003; 163(1): 345-354.
18. Ware L B, Matthay M A. The acute respiratory distress syndrome. N Engl J Med 2000; 342(18): 1334-1349.
19. Noda M, Takeda K, Sugimoto H, et al. Purification and characterization of human fibroblast derived differentiation inducing factor for human monoblastic leukemia cells identical to interleukin-6. Anticancer Res 1991; 11: 961-968.
20. Somers W, Stahl M, Seehra J S. 1.9 A crystal structure of interleukin 6: implications for a novel mode of receptor dimerization and signaling. EMBO J 1997; 16: 989-997.
21. Mucha, Simon R., et al. “Coagulopathy in COVID-19.” Cleveland Clinic Journal of Medicine, Apr. 24, 2020.
22. Zhang T, Sun L X, Feng R E. Comparison of clinical and pathological features between severe acute respiratory syndrome and corona-virus disease 2019; Chin J Tuberc Respir Dis, June 2020, Vol. 43, No. 6.
23. Fox S E, Akmatbekov A, Harbert J L, Li G, Brown J Q, Vander Heide R S. Pulmonary and cardiac pathology in COVID-19: the first autopsy series from New Orleans. MedRxiv 2020; April 10.
24. Levi M, Scully M. How I treat disseminated intravascular coagulation. Blood 2018; 131(8):845-854.
25. Engelmann B, Massberg S. Thrombosis as an intravascular effector of innate immunity. Nat Rev Immunol 2013; 13(1):34-45. [symbol]
26. Kuba K, Imai Y, Rao S, et al. A crucial role of angiotensin converting enzyme 2 (ACE2) in SARS coronavirus-induced lung injury. Nat Med 2005; 11(8):875-87 9. [symbol]
27. Zhang H, Penninger J M, Li Y, Zhong N, Slutsky A. Angiotensin-converting enzyme 2 (ACE2) as a SARS-CoV-2 receptor: molecular mechanisms and potential therapeutic target. Intensive Care Med 2020; 46(4):586-590.
28. Geisbert T W, Hensley L E, Jahrling P B, et al. Treatment of Ebola virus infection with a recombinant inhibitor of factor VIIa/tissue factor: a study in rhesus monkeys. Lancet. 2003; 362(9400):1953-1958.
29. Iba T, Levy JH. Sepsis-induced coagulopathy and disseminated intravascular coagulation. Anesthesiology. 2020; 132(5):1238-1245.
30. Conti P, Ronconi G, Caraffa A, et al. Induction of pro-inflammatory cytokines (IL-1 and IL-6) and lung inflammation by Coronavirus-19 (COVI-19 or SARS-CoV-2): anti-inflammatory strategies. J Biol Regul Homeost Agents. 2020; 34(2):1.
31. Channappanavar R, Zhao J, Perlman S. T cell-mediated immune response to respiratory coronaviruses. Immunol Res. 2014 August; 59(1-3):118-28.
32. Cao X. COVID-19: immunopathology and its implications for therapy. Nat Rev Immunol. 2020 May; 20(5):269-70.
33. Sakr, Y., Giovini, M., Leone, M., Pizzilli, G., Kortgen, A., Bauer, M., Tonetti, T., Duclos, G., Zieleskiewicz, L., Buschbeck, S., Ranieri, V. M., & Antonucci, E. (2020). Pulmonary embolism in patients with coronavirus disease-2019 (COVID-19) pneumonia: A narrative review. Annals of Intensive Care, 10(1).
34. Giannis D, Ziogas I A, Gianni P. Coagulation disorders in coronavirus infected patients: COVID-19, SARS-CoV-1, MERS-CoV and lessons from the past. J Clin Virol. 2020; 127:104362.
35. Oudkerk M, Buller H R, Kuijpers D, van Es N, Oudkerk S F, McLoud T C, et al. Diagnosis, prevention, and treatment of thromboembolic complications in COVID-19: report of the National Institute for Public Health of the Netherlands. Radiology. 2020.
36. Wichmann D, et al. Autopsy Findings and Venous Thromboembolism in Patients with COVID-19. Ann Intern Med. 2020 May; M20-2003.
37. Ackermann M, et al. Pulmonary Vascular Endothelialitis, Thrombosis, and Angiogenesis in Covid-19. N Engl J Med. 2020 July; 383(2):120-8.
38. Helms J, et al.; CRICS TRIGGERSEP Group. High risk of thrombosis in patients with severe SARS-CoV-2 infection: a multicenter prospective cohort study. Intensive Care Med. 2020 June; 46(6):1089-98.
39. Carsana L, et al. Pulmonary post-mortem findings in a series of COVID-19 cases from northern Italy: a two-centre descriptive study. Lancet Infect Dis 2020 Jun. 8 (Epub ahead of print).
40. Oxley T J, et al. Large-Vessel Stroke as a Presenting Feature of Covid-19 in the Young. N Engl J Med. 2020 May; 382(20):e60.
41. Mao L, et al. Neurologic Manifestations of Hospitalized Patients with Coronavirus Disease 2019 in Wuhan, China. JAMA Neurol. 2020 Apr. 10; 77(6):1-9.
42. Cheung C K M, et al Coronavirus Disease 2019 (COVID-19): A Haematologist's Perspective [published online ahead of print, 2020 Jul. 28]. Acta Haematol. 2020; 1-14.
43. Zuo, Y., Estes, S. K., Ali, R. A., Gandhi, A. A., Yalavarthi, S., Shi, H., Sule, G., Gockman, K., Madison, J. A., Zuo, M., Yadav, V., Wang, J., Woodard, W., Lezak, S. P., Lugogo, N. L., Smith, S. A., Morrissey, J. H., Kanthi, Y., & Knight, J. S. (2020). Prothrombotic autoantibodies in serum from patients hospitalized with COVID-19. Science Translational Medicine, eabd3876.
44. Zuo, Y., Shi, H., Li, C., & Knight, J. S. (2020). Antiphospholipid syndrome. Chinese Medical Journal, 133(8), 929-940.
45. Yalavarthi, S., Gould, T. J., Rao, A. N., Mazza, L. F., Morris, A. E., Núñez-Álvarez, C., Hernández-Ramírez, D., Bockenstedt, P. L., Liaw, P. C., Cabral, A. R., & Knight, J. S. (2015). Release of Neutrophil Extracellular Traps by Neutrophils Stimulated With Antiphospholipid Antibodies: A Newly Identified Mechanism of Thrombosis in the Antiphospholipid Syndrome. Arthritis & Rheumatology, 67(11), 2990-3003.
46. Tiekink, Edward R. T., and Jagadese J. Vittal. Frontiers in Crystal Engineering. John Wiley, 2006; pp. 26-27, 46.
47. “L-Theanine Monograph.” Alternative Medicine Review 2005; 10(2):139-147.
48. RSP Amino Acids, LLC., Shirley Mass.
49. Unno T, Suzuki Y, Kakuda T, et al. Metabolism of theanine, a gamma-glutamylethylamide, in rats. J Agric Food Chem 1999; 47:1593-1596.
50. Wang, Dongxu, et al. “Protective Effect and Mechanism of Theanine on Lipopolysaccharide-Induced Inflammation and Acute Liver Injury in Mice.” Journal of Agricultural and Food Chemistry, vol. 66, no. 29, 2018, pp. 7674-7683.
51. Pérez-Vargas, Je, et al. “l-Theanine Prevents Carbon Tetrachloride-Induced Liver Fibrosis via Inhibition of Nuclear Factor KB and down-Regulation of Transforming Growth Factor β and Connective Tissue Growth Factor.” Human & Experimental Toxicology, vol. 35, no. 2, 2015, pp. 135-146.
52. Tsai, Wen-Hsin, et al. “1-Theanine Inhibits Proinflammatory PKC/ERK/ICAM-1/IL-33 Signaling, Apoptosis, and Autophagy Formation in Substance P-Induced Hyperactive Bladder in Rats.” Neurourology and Urodynamics, vol. 36, no. 2, 2016, pp. 297-307.
53. Ali, M., et al. “A Potent Thromboxane Formation Inhibitor in Green Tea Leaves.” Prostaglandins, Leukotrienes and Essential Fatty Acids, vol. 40, no. 4, 1990, pp. 281-283.
54. Giménez-Bastida, J. A., Boeglin, W. E., Boutaud, O., Malkowski, M. G., & Schneider, C. (2019). Residual cyclooxygenase activity of aspirin-acetylated COX-2 forms 15R-prostaglandins that inhibit platelet aggregation. The FASEB Journal, 33(1), 1033-1041.
55. Williams J A, Schacter E. Regulation of macrophage cytokine production by prostaglandin E2. Distinct roles of cyclooxygenase-1 and cyclooxygenase-2. J Biol Chem 1997; 272:25693-25699.
56. Haynes D R, Wright P F, Gadd S J, Whitehouse M W, Vernon-Roberts B. Is aspirin a prodrug for antioxidant and cytokine-modulating oxymetabolites? Agents Actions 1993; 39:49-58.
57. Coulombe, F., Jaworska, J., Verway, M., Tzelepis, F., Massoud, A., Gillard, J., Wong, G., Kobinger, G., Xing, Z., Couture, C., Joubert, P., Fritz, J., Powell, W. and Divangahi, M., 2014. Targeted Prostaglandin E2 Inhibition Enhances Antiviral Immunity through Induction of Type I Interferon and Apoptosis in Macrophages. Immunity, 40(4), pp. 554-568.
58. Koerner, I., Kochs, G., Kalinke, U., Weiss, S., and Staeheli, P. (2007). Protective role of beta interferon in host defense against influenza A virus. J. Virol. 81, 2025-2030.
59. Yin M-J, Yamamoto Y, Gaynor R B. The anti-inflammatory agents aspirin and salicylate inhibit the activity of IkB kinase-b. Nature 1998; 396: 77-80.
60. Claria J, Serhan C N. Aspirin triggers previously undescribed bioactive eicosanoids by human endothelial cell-leukocyte interactions. Proc Natl Acad Sci USA 1995; 92: 9475-9479.
61. Schwenger P, Bellosta P, Vietor I, Basilico C, Skolnik E Y, Vilcek J. Sodium salicylate induces apoptosis via p38 mitogen-activated protein kinase but inhibits tumor necrosis factor-induced c-Jun N-terminal kinase/stress-activated protein kinase activation. Proc Natl Acad Sci USA 1997; 94: 2869-2873.
62. Cronstein B N, Montesinos M C, Weissmann G. Salicylates and sulfasalazine, but not glucocorticoids, inhibit leukocyte accumulation by an adenosine-dependent mechanism that is independent of inhibition of prostaglandin synthesis and p105 of NFkappaB. Proc Natl Acad Sci USA 1999; 96: 6377-6381.
63. Chow J H; Khanna A K; Kethireddy S; Yamane D; Levine A; Jackson A M; McCurdy M T; Tabatabai A; Kumar G; Park P; Benjenk I; Menaker J; Ahmed N; Glidewell E; Presutto E; Cain S; Haridasa N; Field W; Fowler J G; Trinh D; Johnson K N; Kaur A; Lee A; Sebastian K; Ulrich A; Peña S; Carpenter. “Aspirin Use Is Associated with Decreased Mechanical Ventilation, ICU Admission, and In-Hospital Mortality in Hospitalized Patients with COVID-19.” Anesthesia and Analgesia, U.S. National Library of Medicine.
64. Zhou, T., Tsybovsky, Y., Gorman, J., Rapp, M., Cerutti, G., Chuang, G.-Y., Katsamba, P. S., Sampson, J. M., Schön, A., Bimela, J., Boyington, J. C., Nazzari, A., Olia, A. S., Shi, W., Sastry, M., Stephens, T., Stuckey, J., Teng, I-Ting., Wang, P., & Wang, S. (2020). Cryo-EM Structures of SARS-CoV-2 Spike without and with ACE2 Reveal a pH-Dependent Switch to Mediate Endosomal Positioning of Receptor-Binding Domains. Cell Host & Microbe, 28(6), 867-879.e5.
65. Novak, K. M. (ed.). Drug Facts and Comparisons 59th Edition 2005. Wolters Kluwer Health.

St. Louis, Mo. 2005., p. 130.
66. Osol, A. and J. E. Hoover, et al. (eds.). Remington's Pharmaceutical Sciences. 15th ed. Easton, Pa.: Mack Publishing Co., 1975., p. 773.
67. Tham Solution. Tromethamine injection package insert. Lake Forest, Ill.: Hospira; 2005.
68. Hoste E A, Colpaert K, Vanholder R C, Lameire N H, De Waele J J, Blot S I, Colardyn F A. Sodium bicarbonate versus THAM in ICU patients with mild metabolic acidosis. J Nephrol. 2005 May-June; 18(3):303-7. PMID: 16013019.
69. Ritter J M, Doktor H S, Benjamin N. Paradoxical effect of bicarbonate on cytoplasmic pH. Lancet 1990; 335: 1243-6.
70. Levraut J, Giunti C, Ciebiera J P, de Sousa G, Ramhani R, Payan P, Grimaud D. Initial effect of sodium bicarbonate on intracellular pH depends on the extracellular nonbicarbon-ate buffering capacity. Crit Care Med 2001; 29: 1033-9.
71. Goldsmith D J, Forni L G, Hilton P J. Bicarbonate therapy and intracellular acidosis. Clin Sci (Lond) 1997; 93: 593-8.
72. Holmdahl M H, Wiklund L, Wetterberg T, Streat S, Wahlander S, Sutin K, Nahas G. The place of THAM in the management of acidemia in clinical practice. Acta Anaesthesiol Scand 2000; 44: 524-7.
73. Nahas G G, Sutin K M, Fermon C, Streat S, Wiklund L, Wahlander S, Yellin P, Brasch H, Kanchuger M, Capan L, Manne J, Helwig H, Gaab M, Pfenninger E, Wetterberg T, Holmdahl M, Turndorf H. Guidelines for the treatment of acidaemia with THAM. Drugs 1998; 55: 191-224.
74. Lu, C., Leibner, E., & Wright, B. (2016). The use of tris-hydroxymethyl aminomethane in the emergency department. Clinical and Experimental Emergency Medicine, 3(4), 264-265. doi:10.15441/ceem.16.165.
75. Gehlbach B K, Schmidt G A. Bench-to-bedside review: treating acid-base abnormalities in the intensive care unit. The role of buffers. Crit Care 2004; 8:259-65.
76. Luchsinger, P. C. 1961. The use of 2-amino-2-hydroxymethyl-1,3-propanediol in the management of respiratory acidosis. Ann. N.Y. Acad Sci. 92:743-750.
77. Nahas, G. 1959. Use of an organic carbon dioxide buffer in vivo. Science 129:782-783.
78. Kallet R H, Jasmer R M, Luce J M, Lin L H, Marks J D. The treatment of acidosis in acute lung injury with tris-hydroxymethyl aminomethane (THAM). Am J Respir Crit Care Med 2000; 161(4 Pt 1):1149-53.
79. Chand, R., Swenson, E. R., & Goldfarb, D. S. (2020). Sodium bicarbonate therapy for acute respiratory acidosis. Current Opinion in Nephrology & Hypertension, 30(2), 223-230.
80. Irvin, R. T., MacAlister, T. J., & Costerton, J. W. (1981). Tris(hydroxymethyl)aminomethane buffer modification of Escherichia coli outer membrane permeability. Journal of Bacteriology, 145(3), 1397-1403.
81. Moderna COVID19 fact sheet “FACT SHEET FOR RECIPIENTS AND CAREGIVERS.”
82. Reactome|Bicarbonate transporters. (n.d.). Reactome.org. Retrieved Jan. 26, 2022.
83. Romero, M. F., Chen, A., Parker, M. D., & Boron, W. F. (2013). The SLC4 family of bicarbonate transporters. Molecular Aspects of Medicine, 34(2-3), 159-182.
84. Cordat, E., & Casey, J. (2008). Bicarbonate transport in cell physiology and disease. Biochemical Journal, 417(2), 423-439.
85. Gazitua, R. (1979). Factors Determining Peripheral Vein Tolerance to Amino Acid Infusions. Archives of Surgery, 114(8), 897.
86. Marfo K, Garala M, Kvetan V, Gasperino J. Use of Tris-hydroxymethyl aminomethane in severe lactic acidosis due to highly active antiretroviral therapy: a case report. J Clin Pharm Ther. 2009 February; 34(1):119-23. doi: 10.1111/j.1365-2710.2008.00977.x. PMID: 19125910.
87. Interchim Innovations. (n.d.). Buffering agents and Buffers. INTERCHIM.
88. Phosphate-buffered saline (PBS): (2006). Cold Spring Harbor Protocols, 2006(1), pdb.rec8543.
89. Wuhan Desheng Biochemical Technology Co., Ltd. (2021, June 9). China Wuhan Desheng biochemical technology Co., Ltd latest company news about the difference between TRIS buffer and phosphate buffer. Quality Blood Collection Tube Additives & Chemiluminescent Reagent factory from China.
90. Buschmann, M. D., Carrasco, M. J., Alishetty, S., Paige, M., Alameh, M. G., & Weissman, D. (2021). Nanomaterial Delivery Systems for mRNA Vaccines. Vaccines, 9(1), 65.
91. Wolters Kluwer. (2020). Drug facts and comparisons. Lippincott Williams & Wilkins.
92. Liu, X., Li, Z., Liu, S., Chen, Z., Sun, J., Zhao, Z., Huang, Y., Zhang, Q., Wang, J., Shi, Y., Xu, Y., Xian, H., Fang, R., Bai, F., Ou, C., Xiong, B., Lew, A. M., Cui, J., Huang, H., . . . Luo, H. (2020). Therapeutic effects of dipyridamole on COVID-19 patients with coagulation dysfunction. medRxiv.
93. Liu, X., Li, Z., Liu, S., Sun, J., Chen, Z., Jiang, M., Zhang, Q., Wei, Y., Wang, X., Huang, Y.-Y., Shi, Y., Xu, Y., Xian, H., Bai, F., Ou, C., Xiong, B., Lew, A. M., Cui, J., Fang, R., & Huang, H. (2020). Potential therapeutic effects of dipyridamole in the severely ill patients with COVID-19. Acta Pharmaceutica Sinica B, 10(7), 1205-1215.
94. Fata-Hartley, C. L., & Palmenberg, A. C. (2005). Dipyridamole Reversibly Inhibits Mengovirus RNA Replication. Journal of Virology, 79(17), 11062-11070.
95. Huang B, Chen Z, Geng L, Wang J, Liang H, Cao Y, et al. Mucosal profiling of pediatric-onset colitis and IBD reveals common pathogenics and therapeutic pathways. Cell 2019; 179:1160e1176 e24.
96. Insel P A, Murray F, Yokoyama U, Romano S, Yun H, Brown L, et al. cAMP and Epac in the regulation of tissue fibrosis. Br J Pharmacol 2012; 166:447e56.
97. Kruuse, C., Jacobsen, T. B., Lassen, L. H., Thomsen, L. L., Hasselbalch, S. G., Dige-Petersen, H., & Olesen, J. (2000). Dipyridamole Dilates Large Cerebral Arteries Concomitant to Headache Induction in Healthy Subjects. Journal of Cerebral Blood Flow & Metabolism, 20(9), 1372-1379.
98. Mcgonagle, Dennis, et al. “Immune Mechanisms of Pulmonary Intravascular Coagulopathy in COVID-19 Pneumonia.” The Lancet Rheumatology, vol. 2, no. 7, 2020.
99. Goshua, G., Pine, A. B., Meizlish, M. L., Chang, C-Hong., Zhang, H., Bahel, P., Baluha, A., Bar, N., Bona, R. D., Burns, A. J., Dela Cruz, C. S., Dumont, A., Halene, S., Hwa, J., Koff, J., Menninger, H., Neparidze, N., Price, C., Siner, J. M., & Tormey, C. (2020). Endotheliopathy in COVID-19-associated coagulopathy: evidence from a single-centre, cross-sectional study. The Lancet Haematology. ILO International Chemical Safety Cards (ICSC).
100. ILO International Chemical Safety Cards (ICSC).
101. Gammoh, Nour Zahi and Lothar Rink. “Zinc in Infection and Inflammation.” 2017, doi:10.20944/preprints201705.0176.v1.
102. Maret, W. The function of zinc metallothionein: A link between cellular zinc and redox state. J. Nutr. 2000, 130, 1455S-1458S.
103. Mocchegiani, E.; Costarelli, L.; Giacconi, R.; Cipriano, C.; Muti, E.; Malavolta, M. Zinc-binding proteins (metallothionein and alpha-2 macroglobulin) and immunosenescence. Exp. Gerontol. 2006, 41, 1094-1107.
104. Jarosz, M.; Olbert, M.; Wyszogrodzka, G.; Mlyniec, K.; Librowski, T. Antioxidant and anti-inflammatory effects of zinc. Zinc-dependent NF-κB signaling. Inflammopharmacology 2017, 25, 11-24.
105. Liu, M.-J.; Bao, S.; Galvez-Peralta, M.; Pyle, C. J.; Rudawsky, A. C.; Pavlovicz, R. E.; Killilea, D. W.; Li, C.; Nebert, D. W.; Wewers, M. D.; et al. The zinc transporter SLC39A8 is a negative feedback regulator of NF-κB through zinc-mediated inhibition of IKK. Cell Rep. 2013, 3, 386-400.
106. Marreiro, D. D. N.; Cruz, K. J. C.; Morais, J. B. S.; Beserra, J. B.; Severo, J. S.; de Oliveira, A. R. S. Zinc and Oxidative Stress: Current Mechanisms. Antioxidants 2017, 6, 24.
107. Young, B.; Ott, L.; Kasarskis, E.; Rapp, R.; Moles, K.; Dempsey, R. J.; Tibbs, P. A.; Kryscio, R.; McClain, C. Zinc supplementation is associated with improved neurologic recovery rate and visceral protein levels of patients with severe closed head injury. J. Neurotrauma 1996, 13, 25-34.
108. Besecker, B. Y.; Exline, M. C.; Hollyfield, J.; Phillips, G.; DiSilvestro, R. A.; Wewers, M. D.; Knoell, D. L. A comparison of zinc metabolism, inflammation, and disease severity in critically ill infected and noninfected adults early after intensive care unit admission. Am. J. Clin. Nutr. 2011, 93, 1356-1364.
109. Perera, M., El Khoury, J., Chinni, V., Bolton, D., Qu, L., Johnson, P., Trubiano, J., McDonald, C. F., Jones, D., Bellomo, R., Patel, O., & Ischia, J. (2020). Randomised controlled trial for high-dose intravenous zinc as adjunctive therapy in SARS-Cov-2 (COVID-19) positive critically ill patients: Trial protocol. BMJ Open, 10(12), e040580.
110. Zhu H, Rhee J-W, Cheng P, et al. Cardiovascular complications in patients with COVID-19: consequences of viral toxicities and host immune response. Curr Cardiol Rep 2020; 22:32.
111. Feng G, Zheng K I, Yan Q-Q, et al. COVID-19 and liver dysfunction: current insights and emergent therapeutic strategies. J Clin Transl Hepatol 2020; 8:1-7.
112. Fanelli V, Fiorentino M, Cantaluppi V, et al. Acute kidney injury in SARS-CoV-2 infected patients. Crit Care 2020; 24:155. 40.
113. Mubarak M, Nasri H. COVID-19 nephropathy; an emerging condition caused by novel coronavirus infection. J Nephropathol 2020; 9:e21.
114. Castro L, Freeman B A. Reactive oxygen species in human health and disease. Nutrition 2001; 17:161-5.
115. Lachance P A, Nakat Z, Jeong W S. Antioxidants: an integrative approach. Nutrition 2001; 17:835-8.
116. Ruttkay-Nedecky B, Nejdl L, Gumulec J, et al. The role of metallothionein in oxidative stress. Int J Mol Sci 2013; 14:6044-66. 44
117. Prasad A S. Zinc: an antioxidant and anti-inflammatory agent: role of zinc in degenerative disorders of aging. J Trace Elem Med Biol 2014; 28:364-71.
118. te Velthuis A J W, van den Worm S H E, Sims A C, et al. Zn(2+) inhibits coronavirus and arterivirus RNA polymerase activity in vitro and zinc ionophores block the replication of these viruses in cell culture. PLoS Pathog 2010; 6:e1001176.
119. Ferrari E, Wright-Minogue J, Fang J W, et al. Characterization of soluble hepatitis C virus RNA-dependent RNA polymerase expressed in Escherichia coli. J Virol 1999; 73:1649-54.
120. Arnold J J, Ghosh S K, Cameron C E. Poliovirus RNA-dependent RNA polymerase (3D(pol)). Divalent cation modulation of primer, template, and nucleotide selection. J Biol Chem 1999; 274:37060-9.
121. Butterworth B E, Korant B D. Characterization of the large picornaviral polypeptides produced in the presence of zinc ion. J Virol 1974; 14:282-91.
122. Berger M M, Baines M, Raffoul W, et al. Trace element supplementation after major burns modulates antioxidant status and clinical course by way of increased tissue trace element concentrations. Am J Clin Nutr 2007; 85:1293-300.
123. Berger M M, Binnert C, Chiolero R L, et al. Trace element supplementation after major burns increases burned skin trace element concentrations and modulates local protein metabolism but not whole-body substrate metabolism. Am J Clin Nutr 2007; 85:1301-6.
124. Berger M M, Spertini F, Shenkin A, et al. Trace element supplementation modulates pulmonary infection rates after major burns: a double-blind, placebo-controlled trial. Am J Clin Nutr 1998; 68:365-71.
125. Cvijanovich N Z, King J C, Flori H R, Gildengorin G, Vinks A A, Wong H R. Safety and dose escalation study of intravenous zinc supplementation in pediatric critical illness. JPEN J Parenter Enteral Nutr. 2016; 40(6):860-868.
126. Chinni, V., El-Khoury, J., Perera, M., Bellomo, R., Jones, D., Bolton, D., Ischia, J., & Patel, O. (2021). Zinc supplementation as an adjunct therapy for COVID-19: Challenges and opportunities. British Journal of Clinical Pharmacology, 87(10), 3737-3746.

What is claimed is:

1. A method of treating Coronavirus Disease 2019 (COVID-19) of a subject in need thereof, comprising administering an effective amount of aqueous solution that comprises a water-soluble composition comprising: a cocrystal of an acetylsalicylic acid (aspirin) and a L-theanine; and tromethamine.

2. The method of claim 1, wherein the method further comprises administering zinc to the subject.

3. The method of claim 1, wherein the aqueous solution is administered via intravenous infusion.

4. The method of claim 1, wherein the method further comprises administering dipyridamole to the subject.

5. The method of claim 1, wherein the amount of aspirin is between 10 mg and 1.5 g in the intravenous dosage form, the amount of L-theanine is between 10 mg and 1.5 g in the intravenous dosage form, and the amount of tromethamine is between 50 mg and 500 mg in the intravenous dosage form.

6. The method of claim 2, wherein the amount of aspirin is between 10 mg and 1.5 g in the intravenous dosage form, the amount of L-theanine is between 200 mg and 500 mg in the intravenous dosage form, and the amount of tromethamine is between 150 mg and 250 mg in the intravenous dosage form, and the amount of zinc is between 50 mg and 65 mg in the intravenous dosage form.

7. The method of claim 1, wherein the molar ratio of aspirin and L-theanine is 1:1.

8. The method of claim 1, wherein the molar ratio of aspirin, L-theanine, and tromethamine is 1:1: z, wherein z is between about 1 and about 5.

9. The method of claim 1, wherein the weight percentage (wt. %) of the aspirin cocrystal of the water-soluble composition is between about 10% and about 50%.

10. The method of claim 1, wherein the weight percentage (wt. %) of the L-theanine cocrystal of the water-soluble composition is between about 10% and about 50%.

11. The method of claim 1, wherein the weight percentage (wt. %) of tromethamine of the water-soluble composition is between about 10% and about 50%.

12. The method of claim 2, wherein zinc is in the form of zinc chloride.

13. The method of claim 12, wherein the amount of zinc chloride is between about 0.5 mg to about 50 mg in the intravenous dosage form.

14. The method of claim 1, wherein the aqueous solution is isotonic.

15. The method of claim 1, wherein the Coronavirus Disease 2019 (COVID-19) is induced by SARS-CoV-2 selected from the group consisting of Alpha, Beta, Gamma, Delta, Epsilon, Eta, Iota, Kappa, Lambda, Mu, Theta, Zeta, R.1 variant, IHU variant (B.1.640.2), omicron Pango lineage B.1.1.529, omicron descendent Pango lineages BA.1, BA.1.1, BA.2, BA.3, BA.4, and BA.5.

16. The method of claim 4, wherein dipyridamole is administered via the sublingual route.

17. The method of claim 4, wherein dipyridamole is in the form of a clear homogenous solution, suspension, or powder.

18. The method of claim 4, wherein the amount of dipyridamole is between 25 mg and 600 mg.

19. A method of treating coagulopathy of a subject in need thereof, wherein the subject has COVID-19, comprising administering an effective amount of aqueous solution that comprises a water-soluble composition comprising: a cocrystal of an acetylsalicylic acid (aspirin) and a L-theanine; and tromethamine.

20. The method of claim 19, wherein the method further comprises administering zinc to the subject.

21. The method of claim 19, wherein the method further comprises administering dipyridamole to the subject.

22. The method of claim 19, wherein the coagulopathy is a hypercoagulable state.

23. The method of claim 19, wherein the aqueous solution is administered via intravenous infusion.

24. A method of treating endotheliopathy in a subject in need thereof, wherein the subject has COVID-19, comprising administering an effective amount of aqueous solution that comprises a water-soluble composition comprising: a cocrystal of an acetylsalicylic acid (aspirin) and a L-theanine; and tromethamine.

25. The method of claim 24, wherein the method further comprises administering zinc to the subject.

26. The method of claim 24, wherein the method further comprises administering dipyridamole to the subject.

27. The method of claim 24, wherein the aqueous solution is administered via intravenous infusion.
